(19) United States
(12) Patent Application Publication
(10) Pub. No.: US 2025/0375462 A1
(43) Pub. Date: Dec. 11, 2025

(54) NUTRACEUTICAL COMPOSITION FOR COMBATING AGING IN HUMAN AND NON-HUMAN ANIMALS AND BEVERAGES AND/OR FOOD PRODUCTS AND/OR FOOD SUPPLEMENTS CONTAINING THE SAME

(71) Applicants: DOUGLAS JAY SPIEL, PERRINEVILLE, NJ (US); RAYMOND ANTHONY JOAO, YONKERS, NY (US)

(72) Inventors: DOUGLAS JAY SPIEL, PERRINEVILLE, NJ (US); RAYMOND ANTHONY JOAO, YONKERS, NY (US)

(21) Appl. No.: 19/228,537

(22) Filed: Jun. 4, 2025

Related U.S. Application Data
(60) Provisional application No. 63/658,824, filed on Jun. 11, 2024.

(51) Int. Cl. A61K 31/706; A61K 31/05; A61K 31/09; A61K 31/132; A61K 31/19; A61K 31/194; A61K 31/198; A61K 31/205; A61K 31/275; A61K 31/352; A61K 31/353; A61K 31/37; A61K 31/4375; A61K 31/4525; A61K 31/522; A61K 31/7008; A61K 31/728; A61K 31/737; A61K 33/00; A61K 36/185; A61K 36/41; A61K 36/54; A61K 36/9066; A61K 36/9068; A61K 38/06

(52) U.S. Cl. CPC A61K 31/706; A61K 31/05; A61K 31/09; A61K 31/132; A61K 31/19; A61K 31/194; A61K 31/198; A61K 31/205; A61K 31/275; A61K 31/352; A61K 31/353; A61K 31/37; A61K 31/4375; A61K 31/4525; A61K 31/522; A61K 31/7008; A61K 31/728; A61K 31/737; A61K 33/00; A61K 36/41; A61K 36/54; A61K 36/5777; A61K 36/9066; A61K 36/9068; A61K 38/063

(57) ABSTRACT

A nutraceutical composition, consisting of Nicotinamide Mononucleotide in a range 250-1000 mg, Spermidine in a range 0.5-10 mg, Rhodiola Rosea in a range 200-600 mg, Cacao in a range 1000-10,000 mg, L-Theanine in a range 50-200 mg, Glucosamine in a range 500-3000 mg, and Hyaluronic acid in a range 50-240 mg, or consisting of Epigallocatechin-3-gallate in a range 100-500 mg, Quercetin in a range 250-1000 mg, Resveratrol in a range 150-1000 mg, Sulforaphane in a range 1-50 mg, and Calcium Alphaketoglutarate in a range 300-2000 mg, or consisting of Microdose Lithium in a range 1-5 mg, Trimethylglycine in a range 500-2000 mg, Glutathione in a range 250-1000 mg, Fisetin in a range 100-1000 mg, Urolithin A in a range 250-2000 mg, Sodium Butyrate in a range 100-3000 mg, Berberine in a range 500-1500 mg, Apigenin in a range 50-500 mg, Cinnamon in a range 1000-6000 mg, and Malate in a range 1000-6000 mg.

### RELATED APPLICATIONS

This application claims the benefit of the priority of U.S. Provisional Patent Application Ser. No. 63/658,824, filed Jun. 11, 2024, and entitled “NUTRACEUTICAL COMPOSITION FOR COMBATING AGING IN HUMAN AND NON-HUMAN ANIMALS AND BEVERAGES AND/OR FOOD PRODUCTS AND/OR FOOD SUPPLEMENTS CONTAINING THE SAME”, the subject matter and teachings of which are hereby incorporated by reference herein in their entirety.

### FIELD OF THE INVENTION

The present invention pertains to a nutraceutical composition for combating aging in human and non-human animals and beverages, and/or food products, and/or food supplements, containing the same and, in particular, the present invention pertains to a nutraceutical composition for combating aging in human and non-human animals and beverages, and/or food products, and/or food supplements, containing the same which contain a combination of nutraceutical substances and/or products which address, and/or which subserve and/or which can serve to promote and/or facilitate the combating of, each one of twelve identified pillars of aging.

### BACKGROUND OF THE INVENTION

While the field of aging is an evolving science, it is clear that normal aging inevitably leads to age-related diseases. It is, therefore, of paramount importance to slow the aging process to stave off these afflictions. Clinicians and scientists have attempted to quantify the deficiencies of aging into discrete pillars so that the they can be better characterized and understood. There are many food and drink products, including, but not limited to, various beverage products, powdered products, chewable products, and/or foods products, which are offered in the marketplace which are touted as having various health benefits, nutritional benefits, energy enhancing benefits, and even anti-aging benefits. It is submitted, however, that while these various products might provide certain benefits, they still lack in their ability to provide longevity or to provide optimal longevity.

While various products, such as certain nutraceutical products, are known and available in the marketplace, it is submitted that no one particular nutraceutical product can effectively combat all of the various pillars of aging, and it is submitted that all of the pillars of aging must be combated in order to maximize an individual's longevity.

It is submitted that there are twelve pillars of aging which must be addressed in order to follow a proper longevity nutrition regime. It is submitted that the twelve pillars of aging include: 1) genomic instability; 2) telomere attrition; 3) epigenetic alterations; 4) loss of proteostasis; 5) deregulated nutrient sensing; 6) mitochondrial dysfunction; 7) cellular senescence; 8) stem cell exhaustion; 9) altered intercellular communication; 10) extracellular matrix stiffness; 11) altered cellular metabolism/autophagy; and 12) inflammation.

It is submitted that there currently exists no product in the marketplace which can improve and/or maximize one's longevity which effectively addresses and effectively combats all of the aforementioned twelve pillars of aging. Further, it is submitted that there are currently no products in the marketplace which utilize various nutraceutical products and ingredients in sufficient quantities necessary to, provide, in a single serving, a longevity product which effectively addresses and effectively combats the aforementioned twelve pillars of aging.

As a result, consumers are forced to pour through numerous online companies, vitamin stores, and pharmaceutical supply companies, in order to obtain each of the various and individual supplements needed, and/or to piecemeal combine them together, in order to create their own homemade longevity cocktails or food products. It is also submitted that even those self-proclaimed providers of products or ingredients for combating aging typically require consumers to further combine these products or ingredients with other products or ingredients which can result in cocktails or food products which have insufficient dosages to effectively combat aging and the twelve pillars of aging. Further, the average consumer typically lacks the requisite knowledge to create their own blend of nutraceutical products and ingredients in order to address and effectively combat the twelve pillars of aging.

In view of the foregoing, it is submitted that no nutraceutical composition exists in the marketplace which addresses and combats all of the twelve pillars of aging in an effective and efficient manner.

It is further submitted that yet another problem lies in providing for the most effective and efficient use of nutraceutical products and/or nutraceutical compositions and in providing for an effective and efficient administration of the same to an individual. It is submitted that rates of absorption, time of day optimal effectiveness, and metabolic interactions between and among various nutraceutical products, can all play a factor in providing for the effective and efficient use of nutraceutical products and/or in achieving optimal results from the use of the same by individuals.

### SUMMARY OF THE INVENTION

The present invention pertains to a nutraceutical composition for combating aging in human and non-human animals and beverages, and/or food products, and/or food supplements, containing the same and, in particular, the present invention pertains to a nutraceutical composition for combating aging in human and non-human animals and beverages, and/or food products, and/or food supplements containing the same, which contain a combination of nutraceutical substances and/or products which address, and/or which subserve and/or which can serve to promote and/or facilitate the combating of, each one of twelve identified pillars of aging, which overcome the shortcomings and shortfalls of prior art compositions and substances.

The present invention is, and provides, a nutraceutical composition which includes, but which is not limited to, at least the following 17 nutraceutical ingredients (referred to hereinafter as the “17 core nutraceutical ingredients”): 1) Berberine; 2) Trimethylglycine (TMG); 3) Microdose Lithium; 4) Epigallocatechin-3-gallate (EGCG); 5). Quercetin; 6) Fisetin; 7). Turmeric/Curcumin; 8). Resveratrol; 9). Glutathione; 10) Nicotinamide Mononucleotide (NMN) or Nicotinamide Riboside (NR); 11) Calcium Alphaketoglutarate (CaAKG); 12) Spermidine; 13) Urolithin A; 14) Sulforaphane; 15) Glucosamine; 16) Hyaluronic acid; and 17) Sodium Butyrate.

It is submitted that some of the foregoing 17 core nutraceutical ingredients may yield enhanced absorption in a liposomal preparation such as, for example, glutathione. It is also submitted that some of the 17 core nutraceutical ingredients, along with other nutraceutical ingredients, demonstrate enhanced absorption with co-administration of other compounds such as, for example, curcumin with black pepper (which contains Piperine) and sulforaphane with myrosinase.

It also submitted that many of the nutraceutical ingredients noted above and further listed hereinafter are known to expand lifespan in certain organisms.

At least two or more of the each of the above listed 17 core nutraceutical ingredients can address and combat each of the twelve pillars of aging. In this regard, the 17 core nutraceutical ingredients listed herein can ensure appropriate coverage for addressing and combating each of the twelve pillars of aging.

Any other suitable nutraceutical compounds or nutraceutical ingredients can also be utilized in the present invention in addition to the 17 core nutraceutical ingredients listed above. These additional nutraceutical compounds or nutraceutical ingredients can include, but are not limited to: 1) Pterostilbene; 2) Rhodiola Rosea; 3) Malate (or Malic Acid); 4) L-Theanine; 5) Ginger; 6) Cinnamon; and/or 7) Apigenin.

The nutraceutical composition of the present invention can be manufactured by mixing the above-identified 17 core nutraceutical ingredients, together and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, protein drinks, and/or any other edible food or beverages which can contain supplements such as those of, and/or provided by, the present invention.

The nutraceutical composition of the present invention can also be manufactured by combining, blending, or mixing, together the above-identified 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, by mixing and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, and/or any other edible food or beverage which can contain supplements such as those of, and/or provided by, the present invention.

For example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with water, or with any suitable juice or drink, in order to obtain a nutraceutical-base product for providing longevity.

As and for another example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with green tea in order to obtain a nutraceutical-based green tea product for providing longevity.

As and for another example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with black tea in order to obtain a nutraceutical-based black tea product for providing longevity.

As and for still another example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with any type or kind of tea in order to obtain a nutraceutical-based tea product for providing longevity.

As and for yet another example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with any type or kind of coffee, which can be caffeinated coffee, de-caffeinated coffee, or any flavored coffee, in order to obtain a nutraceutical-based coffee product for providing longevity.

As and for another example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with any type or kind of red wine in order to obtain a nutraceutical-based red wine beverage or drink product for providing longevity.

As and for still another example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with any type or kind of diet beverage or drink, nutrition beverage or drink, energy beverage or drink, protein beverage or drink, or any other type or kind of beverage or drink, in order to obtain a nutraceutical-based beverage or drink product for providing longevity.

As and for yet another example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed together in powder form and/or in liquid form, and/or in any combination of the same, and the resulting mixture can be used in producing a chewable food product for providing longevity.

All of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can also be mixed together in powder form and/or in liquid form, and/or in any combination of the same, and the resulting mixture can be used in producing a food product such as food bar, a snack bar, a cookie, a syrup, a drink or beverage additive or supplement, or a food additive or food supplement which can be in liquid form or in powdered form, for providing longevity.

A process for producing the nutraceutical composition of the present invention and/or the beverages, and/or food products, and/or food supplements, containing the same, can be performed by combining, mixing, or blending, in any appropriate order or manner, the above-identified 17 core nutraceutical ingredients, including Berberine, Trimethylglycine, Microdose Lithium; Epigallocatechin-3-gallate (EGCG), Quercetin, Fisetin, Turmeric/Curcumin, Resveratrol, Glutathione, Nicotinamide Mononucleotide (NMN) or Nicotinamide Riboside (NR), Calcium Alphaketoglutarate, Spermidine, Urolithin A, Sulforaphane, Glucosamine, Hyaluronic acid, and Sodium Butyrate, each of which can be in a powdered form and/or in a liquid form, and each of which can be in respective amounts per servings as provided in Table 1, in an appropriate container or containment system.

One or more of the 17 core nutraceutical ingredients can also be placed in a liposomal preparation, such as, for example, one containing glutathione. One or more of the 17 core nutraceutical ingredients can utilize a co-agent which can include curcumin with black pepper (which contains Piperine) and/or sulforaphane with myrosinase.

Any other or additional nutraceutical ingredient(s), such as Pterostilbene, Rhodiola Rosea, Malate (or Malic Acid), L-Theanine, Ginger; Cinnamon, and/or Apigenin, and/or any other suitable nutraceutical ingredient(s), can also be added to the above-described mixture.

The nutraceutical ingredients can thereafter be mixed together by mixing and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, and/or any other edible food or beverage which can contain supplements such as those of, and/or provided by, the present invention.

The nutraceutical ingredients which are utilized can be mixed together so as to yield the resulting nutraceutical composition of the present invention. The resulting nutraceutical composition can be in liquid form or in powdered form. If a powdered form is desired from a resulting liquid nutraceutical composition, an appropriate de-hydration process or operation can be performed in the container or containment system and/or in a second or other container or containment system.

Thereafter, the resulting nutraceutical composition can be in liquid form or in powdered form can be utilized in any appropriate manner, such as in producing a longevity nutraceutical-based beverage, a longevity nutraceutical-based drink, a longevity nutraceutical-based chewable food product, a longevity nutraceutical-based food product such as food bar, a snack bar, a cookie, or a syrup, a longevity nutraceutical-based drink or beverage additive or supplement, or a longevity nutraceutical-based food additive or food supplement.

The respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement, can be produced using the nutraceutical composition (also referred to as the “resulting nutraceutical composition”) which was produced. The respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement, can be produced using any respective, suitable, and/or appropriate method(s), technique(s), process(es), and/or operation(s) known by those skilled in the art of food production or manufacturing, beverage or drink production or manufacturing, food supplement production or manufacturing, and/or beverage or drink supplement production or manufacturing.

Any natural flavoring(s) and/or any artificial flavoring(s) can also be added to the respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement.

The present invention is, and provides, a nutraceutical composition which also can include a reduced number of nutraceutical ingredients while still providing for the addressing and the combating of all twelve of the pillars of aging. In one such preferred embodiment, thirteen or more nutraceutical ingredients can be utilized which can be selected from among the following 24 nutraceutical ingredients: 1) Berberine; 2) Trimethylglycine (TMG); 3) Microdose Lithium; 4) Epigallocatechin-3-gallate (EGCG); 5). Quercetin; 6) Fisetin; 7). Turmeric/Curcumin; 8) Resveratrol; 9). Glutathione; 10) Nicotinamide Mononucleotide (NMN) or Nicotinamide Riboside (NR); 11) Calcium Alphaketoglutarate (CaAKG); 12) Spermidine; 13) Urolithin A; 14) Sulforaphane; 15) Glucosamine; 16) Hyaluronic acid; 17) Sodium Butyrate; 18) Pterostilbene; 19) Rhodiola Rosea; 20) Malate (or Malic Acid); 21) L-Theanine; 22) Ginger; 23) Cinnamon; and 24) Apigenin. The above 24 nutraceutical ingredients, along with suggested dosages per serving of nutraceutical composition, are listed in TABLE 2.

Less than thirteen of the foregoing 24 nutraceutical ingredients, or any other nutraceutical ingredients not identified herein, can also be utilized provided the nutraceutical composition formed thereby can effectively address and combat all twelve of the pillars of aging in an effective manner.

At least the following thirteen nutraceutical ingredients can be utilized in producing the nutraceutical composition of the present invention: 1) Berberine; 2) Trimethylglycine (TMG); 3) Microdose Lithium; 4) Epigallocatechin-3-gallate (EGCG); 5) Fisetin; 6) Resveratrol; 7) Glutathione; 8) Nicotinamide Mononucleotide (NMN) or Nicotinamide Riboside (NR); 9) Calcium Alphaketoglutarate (CaAKG); 10) Spermidine; 11) Urolithin A; 12) Hyaluronic acid; and 13) Sodium Butyrate.

Additionally, one or more of any other nutraceutical ingredients identified herein, or any other appropriate and/or suitable nutraceutical ingredient(s) not described herein, can also be utilized along with the above 13 listed nutraceutical ingredients.

At least the above listed 13 nutraceutical ingredients, can be utilized to provide for a minimal or a reduced number of nutraceutical ingredients which can effectively address and combat all twelve of the pillars of aging. All of the at least 13 nutraceutical ingredients, and/or any one or more of the other herein-mentioned additional nutraceutical compounds or nutraceutical ingredients, and/or any other suitable or appropriate nutraceutical compounds or nutraceutical ingredients, can also be mixed together in powder form and/or in liquid form, and/or in any combination of the same, and the resulting mixture can be used in producing a food product such as food bar, a snack bar, a cookie, a syrup, a drink or beverage additive or supplement, or food additive or food supplement which can be in liquid form or in powdered form, for providing longevity.

A process for producing the nutraceutical composition of the present invention and/or the beverages, and/or food products, and/or food supplements, containing the same, can be performed by combining, mixing, or blending, in any appropriate order or manner, the at least 13 nutraceutical ingredients, along with any other nutraceutical ingredient(s), if utilized, each of which can be in a powdered form and/or in a liquid form, and each of which can be in respective amounts per servings as provided in Table 1 or in Table 2, in an appropriate container or containment system.

One or more of the nutraceutical ingredients can also be placed in a liposomal preparation, such as, for example, one containing glutathione. One or more of the nutraceutical ingredients can also utilize a co-agent which can include curcumin with black pepper (which contains Piperine) and/or sulforaphane with myrosinase.

The nutraceutical ingredients can thereafter be mixed together by mixing and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, and/or any other edible food or beverage which can contain supplements such as those of, and/or provided by, the present invention.

As noted above, the nutraceutical ingredients which are utilized can be mixed together so as to yield the resulting nutraceutical composition of the present invention. As is also noted above, the resulting nutraceutical composition can be in liquid form or in powdered form. If a powdered form is desired from a resulting liquid nutraceutical composition, an appropriate de-hydration process or operation can be performed in the container or containment system and/or in a second or other container or containment system.

The nutraceutical composition of the present invention can also contain the nutraceutical Fucoidan in a range of 100-4000 mg. The Fucoidan can be an additional ingredient in any nutraceutical composition of, and prepared pursuant to, the present invention. Fucoidan can be used as a suitable substitute for any ingredient in any nutraceutical composition of, and prepared pursuant to, the present invention.

The nutraceutical composition of the present invention can also contain the nutraceutical Cacao in any suitable and safe amount or range provided that said amount does not exceed any safe or recommended daily dosage amount or range. The Cacao can be an additional ingredient in any nutraceutical composition of, and prepared pursuant to, the present invention. Cacao can be used as a suitable substitute for any ingredient in any nutraceutical composition of, and prepared pursuant to, the present invention.

The nutraceutical composition of the present invention can be manufactured and sold in powder form as a powder which can be added to, and/or mixed with, any drink or beverage, or which can be added to, and/or mixed with, any food. The nutraceutical composition of the present invention, which can be manufactured as a powder, can be flavored with any appropriate and/or suitable flavor or flavoring, which flavor or flavoring can be, or can include, a natural flavor or flavoring or any combination of natural flavors or flavorings.

The nutraceutical composition of the present invention, which can be manufactured as a powder, can be flavored with any appropriate and/or suitable flavor or flavoring, which flavor or flavoring can be, or can include, an artificial flavor or flavoring or any combination of artificial flavors or flavorings.

The nutraceutical composition of the present invention, which can be manufactured as a powder, can be flavored with any appropriate and/or suitable flavor or flavoring, which flavor or flavoring can be, or can include, any combination of a natural flavor or flavoring and an artificial flavor or flavoring or any combination of an artificial flavor or flavoring and one or more natural flavor(s) or flavoring(s) or any combination of a natural flavor or flavoring and one or more artificial flavor(s) or flavoring(s).

The nutraceutical composition of the present invention, which can be manufactured as a powder, can also be non-flavored.

The nutraceutical composition of the present invention, which can be manufactured as a powder, can also produced with caffeine as a caffeinated product. The nutraceutical composition of the present invention, which can be manufactured as a powder, can also produced with without caffeine as a non-caffeinated product or as a de-caffeinated product.

The nutraceutical composition of the present invention can also be produced or manufactured as a concentrated liquid or as a syrup which can be added to, and/or mixed with, any drink or beverage, or which can be added to, and/or mixed with, any food. The nutraceutical composition of the present invention, which can also be produced as a concentrated liquid or as a syrup, can be flavored with any natural flavor or flavoring, or can be flavored with any artificial flavor or flavoring, or can be flavored with any combination of one or more natural flavor(s) or flavoring(s) and one or artificial flavor(s) or flavoring(s). The nutraceutical composition of the present invention, which can also be produced as a concentrated liquid or as a syrup, can be unflavored. The herein-described nutraceutical composition of the present invention, which can also be produced as a concentrated liquid or as a syrup, can be caffeinated or can be uncaffeinated or de-caffeinated.

The herein-identified nutraceutical ingredients, including, but not limited to, Berberine, Trimethylglycine (TMG), Microdose Lithium, Epigallocatechin-3-gallate (EGCG), Fisetin, Resveratrol, Glutathione, Nicotinamide Mononucleotide (NMN) or Nicotinamide Riboside (NR), Calcium Alphaketoglutarate (CaAKG), Spermidine, Urolithin A, and Hyaluronic acid, or any combination of any of the herein-identified nutraceutical ingredients, which can include any of the ingredients identified in Table 2 and/or Fucoidan and/or cacao, can also be utilized to provide for a nutraceutical composition which can effectively address and combat all twelve of the pillars of aging.

The nutraceutical composition of the present invention can be mixed and/or processed in order to yield a nutraceutical composition in a powder form or powdered form which can be added to, or mixed into and/or with, water or any suitable liquid drink or beverage, or a juice, coffee, tea, or any other type or kind of beverage. The nutraceutical composition of the present invention can be mixed and/or processed in order to yield a nutraceutical composition in a powder form or powdered form which can be added to, or mixed into and/or with, any cold liquid or beverage or any hot or warm liquid of beverage.

For example, the nutraceutical composition in powder form or powdered form can be packaged in a stick package, in a stick packet, or/and in any suitable packet or packaging. Such a packaged nutraceutical composition provided in powder form can be mixed into or with any water or other liquid or beverage.

The nutraceutical composition of the present invention can be mixed and/or processed in order to yield a nutraceutical composition in a liquid form or in a syrup form, which nutraceutical composition can be added to, or mixed into and/or with, water or any suitable liquid drink or beverage, or a juice, coffee, tea, or any other type or kind of beverage.

The nutraceutical composition of the present invention, in a liquid form or in a syrup form, can be added to, or mixed into and/or with, any cold liquid or beverage or any hot or warm liquid of beverage. For example, the nutraceutical composition in liquid form or in syrup form can be packaged in any suitable container, bottle, or can, made of any suitable material. In a preferred embodiment, such a packaged nutraceutical composition, in liquid form or in syrup form can be mixed into or with any water or other liquid or beverage.

The herein-identified nutraceutical ingredients, and/or Fucoidan and/or cacao, can be utilized to provide for a nutraceutical composition which can effectively address and combat all twelve of the pillars of aging.

The nutraceutical composition of the present invention can be mixed and/or processed in order to yield a nutraceutical composition in a powder form or powdered form which can be added to, or mixed into and/or with, water or any suitable liquid drink or beverage, or a juice, coffee, tea, or any other type or kind of beverage. The nutraceutical composition of the present invention can be mixed and/or processed in order to yield a nutraceutical composition in a powder form or powdered form which can be added to, or mixed into and/or with, any cold liquid or beverage or any hot or warm liquid of beverage. For example, the nutraceutical composition in powder form or powdered form can be packaged in a stick package, in a stick packet, or/and in any suitable packet or packaging. Such a packaged nutraceutical composition provided in powder form can be mixed into or with any water or other liquid or beverage.

The nutraceutical composition of the present invention can be mixed and/or processed in order to yield a nutraceutical composition in a liquid form or in a syrup form which nutraceutical composition can be added to, or mixed into and/or with, water or any suitable liquid drink or beverage, or a juice, coffee, tea, or any other type or kind of beverage. The nutraceutical composition of the present invention, in a liquid form or in a syrup form, can be added to, or mixed into and/or with, any cold liquid or beverage or any hot or warm liquid of beverage. The nutraceutical composition in liquid form or in syrup form can be packaged in any suitable container, bottle, or can, made of any suitable material. Such a packaged nutraceutical composition, in liquid form or in syrup form can be mixed into or with any water or other liquid or beverage.

Thereafter, the resulting nutraceutical composition can be in liquid form or in powdered form can be utilized in any appropriate manner, such as in producing a longevity nutraceutical-based beverage, a longevity nutraceutical-based drink, a longevity nutraceutical-based chewable food product, a longevity nutraceutical-based food product such as food bar, a snack bar, a cookie, or a syrup, a longevity nutraceutical-based drink or beverage additive or supplement, or a longevity nutraceutical-based food additive or food supplement.

The respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement, can be produced using the nutraceutical composition which was produced. The respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement, can be produced using any respective, suitable, and/or appropriate method(s), technique(s), process(es), and/or operation(s) known by those skilled in the art of food production or manufacturing, beverage or drink production or manufacturing, food supplement production or manufacturing, and/or beverage or drink supplement production or manufacturing.

As also noted herein, any natural flavoring(s) and/or any artificial flavoring(s) can also be added to the respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement.

As noted above, the nutraceutical composition of the present invention can also be produced from less than thirteen nutraceutical ingredients, with the minimum number only being limited depending upon the effective addressing and effective combating of the all twelve of the pillars of aging.

The nutraceutical composition of the present invention can be used to provide a pre-packaged beverage or drink, a pre-packaged tea beverage or drink, a pre-packaged coffee beverage or drink, a pre-packaged red wine beverage or drink, a pre-packaged carbonated beverage or drink, a pre-packaged caffeinated beverage or drink, a product which can be offered in powered powdered form, a chewable food product, a food product, a food snack bar product, a food supplement product which can be provided in liquid form or in powdered form, and/or any other consumable beverage, drink, edible product, food product, or food supplement. In a preferred embodiment, the present invention can provide a nutraceutical composition which can be consumed in any beverage, drink, edible, food product, or food supplement, daily by itself as a stand alone supplement and/or as a daily solution to address and combat the problem of aging while dispensing with the need to obtain and mix the individual ingredients prior to consumption.

The present invention can provide a food product containing the nutraceutical composition which can be a food supplement which can be added to a food, a beverage, or a drink.

The present invention can provide a food product containing the nutraceutical composition which can be a food supplement which can be applied to a food, a beverage, or a drink. The food product or food supplement of the present invention can also be sprinkled or poured on or over, or into, any food, beverage, or drink.

It is submitted that the nutraceutical composition of the present invention and any products containing the same can provide benefits in and to all major organ systems. These benefits could include, but are not limited to, improved metabolic health, improved cardiovascular health, decreased inflammation, enhanced anti-oxidant benefits, cancer prevention, improved gastrointestinal health, neuroprotection, mood stabilization, improved cognitive health, improved brain health, improved and enhanced antibacterial and anti-viral support, improved and enhanced immune system support, improved respiratory health, improved and enhanced exercise performance and recovery, improved skin health, improved joint health, improved liver health, improved blood sugar control, improved weight management, improved detoxification, improved cellular health and repair, improved energy metabolism, improved protein synthesis, improved wound healing, improved cellular health, improved mitochondrial health, and improved and enhanced life span extension.

The nutraceutical composition of the present invention serves to address and combat the twelve pillars of aging. Further, the nutraceutical composition of the present invention serves to address and combat the twelve pillars of aging in an efficient and effective manner.

Applicant hereby incorporates by reference herein all processes, proprietary and non-proprietary, which are known to those skilled in the pertinent and respective art(s), as of the date of the filing of this patent application, in order to perform any of the method(s), technique(s), process(es), and/or operation(s), needed or desired in performing any of the food production or manufacturing, the beverage or drink production or manufacturing, the food supplement production or manufacturing, and/or the beverage or drink supplement production or manufacturing, in producing any beverages, drinks, foods, and/or food supplements, described herein as being produced using any of the nutraceutical compositions of the present invention.

Applicant also hereby incorporates by reference herein the subject matter and teachings of U.S. Pat. No. 11,839,622, entitled “CONSUMABLE NUTRACEUTICAL COMPOSITION”, the subject matter and teachings of which are incorporated by reference herein in their entirety, as if to be fully restated herein, so as incorporate the teachings of same, and so as to improve upon the teachings of the same.

The nutraceutical composition of the present invention can also be produced so as to include any types or kinds of ingredients typically found in energy drinks, protein drinks, vitamin drinks, energy foods, protein foods, vitamin-enriched foods, energy food and/or beverage supplements, protein food and/or beverage supplements, and/or vitamin food and/or beverage supplements.

The present invention can be, and/or can include, multiple nutraceutical compositions which can each contain different nutraceutical ingredients, and which can be designed to be administered to, taken by, or ingested by, an individual at different times of the day. It is submitted that various nutraceutical products or ingredients can have absorption rates, can interact differently with, and/or can impact metabolic rates, and/or can have other effects, which can differ depending upon the time of the day at which they are ingested, and that the same can differ depending upon any other nutraceutical products or ingredients which are ingested at the same time.

It is also submitted that the specific properties of each nutraceutical product or ingredient should be considered when seeking to effectuate optimal absorption and synergies when taking the nutraceutical product or ingredient along with another nutraceutical product or ingredient or with other nutraceutical products or ingredients.

The present invention can be, and/or can include, multiple nutraceutical compositions, each of which can be designed to be taken by an individual, or administered to an individual, at different times of day in order to facilitate optimal effectiveness in use. Each of the multiple nutraceutical compositions of the present invention can be selected for optimal use at its respective time of ingestion.

The present invention can be, and/or can include, at least three nutraceutical compositions, wherein, in a case of three nutraceutical compositions, a first nutraceutical composition is ingested in the morning, a second nutraceutical composition is ingested at mid-day, and a third nutraceutical composition is ingested in the evening. The nutraceutical ingredients selected for use in each of the respective first nutraceutical composition, second nutraceutical composition, and the third nutraceutical composition, can be selected for their respective properties and effects.

The present invention can also be, and/or can include, two or more, or any number of, nutraceutical compositions, each of which can be ingested at particular and/or specified times of day in order to provide optimal effectiveness, and/or optimal absorption and/or synergies. In this regard, it is submitted that rates of absorption, time of day optimal effectiveness, and metabolic interactions between and among various nutraceutical products, can all play a factor in providing for an effective and efficient use of nutraceutical products and/or in achieving optimal results from the use of the same by individuals. The present invention can include multiple nutraceutical compositions, with each being taken or ingested at particular times of the day, such as, for example, but not limited to, in the morning, at noon or at midday, or in the evening, and/or at any other time or times and/or pursuant to any suitable or appropriate schedule.

In an embodiment, wherein the present invention is directed to at least three nutraceutical compositions, each of which can be taken or ingested at at least three separate times of the day which can be in the morning, at noon or at midday, or in the evening, respectively, and each of the exemplary nutraceutical compositions can include any combination of the following nutraceutical ingredients or nutraceuticals:

### I. First Nutraceutical Composition (Morninm Composition):

A first nutraceutical composition, which can be taken or ingested in the morning, can include any one or more of, or any combination of, the following:
1) NMN (Nicotinamide Mononucleotide); 2) Spermidine; 3) Rhodiola Rosea; 4) Turmeric; 5) Ginger; 6) Cacao; 7) L-Theanine; 8) Glucosamine; and 9) Hyaluronic Acid.

### II. Second Nutraceutical Composition (Noon or Midday Composition):

A second nutraceutical composition, which can be taken or ingested at noon or at midday, can include any one or more of, or any combination of, the following:
1) EGCG (Epigallocatechin Gallate); 2) Quercetin; 3) Resveratrol; 4) Sulforaphane; and 5) Calcium Alpha-Ketoglutarate.

### III. Third Nutraceutical Composition (Evening Composition):

A third nutraceutical composition, which can be taken or ingested in the evening, can include any one or more of, or any combination of, the following:
1) Microdose Lithium; 2) Trimethylglycine (TMG); 3) Glutathione; 4) Fisetin; 5) Urolithin A; 6) Sodium Butyrate; 7) Berberine; 8) Apigenin; 9) Cinnamon; and 10) Malate.

In addition to the foregoing, each of the above nutraceutical compositions, as well as each of any and/or all of the various nutraceutical compositions disclosed herein, can also include antioxidants, fat soluble supplements, and/or pro-energetic supplements, in order to provide other benefits. As and for an example, antioxidants, such as, but not limited to, Quercetin, Resveratrol, and Fisetin, can also be included in, and/or taken together, in a nutraceutical composition as they can support each other in combating oxidative stress. In this regard, any nutraceutical composition disclosed herein can also include Quercetin, Resveratrol, and Fisetin.

As and for another example, fat soluble supplements, such as, but not limited to, Turmeric and Resveratrol, should be taken or ingested with meals containing fats for better absorption. In this regard, any nutraceutical composition disclosed herein which can be taken or ingested with or in the vicinity of a meal containing fats can also include Turmeric and/or Resveratrol and/or any other fat-soluble supplement(s).

As and for another example, pro-energetic supplements, such as, but not limited to, Nicotinamide Mononucleotide (NMN), Spermidine, and Calcium Alphaketoglutarate (CaAKG), can generally be best taken or ingested during the first half of the day so as to be best aligned with the body's natural peak in metabolic activities. In this regard, any nutraceutical composition disclosed herein which can be taken or ingested in or during the first half of the day can also include Nicotinamide Mononucleotide (NMN), Spermidine, and Calcium Alphaketoglutarate (CaAKG), and/or any other pro-energetic supplement(s).

Any of the herein-described and/or herein-identified nutraceutical compositions of the present invention can also include or contain Pterostilbene which is submitted to be a compound which is similar to Resveratrol and having antioxidant properties as well as anti-inflammatory properties. It is also submitted that, since Pterostilbene can have a potential impact on cognitive function and overall health, Pterostilbene can be taken or ingested, in or with a nutraceutical composition(s) of the present invention, in the morning or at noon or at midday so as to more efficiently and more effectively utilizing its energizing effects throughout the day as well as possible or potentially aiding in maintaining focus and/or vitality.

Any of the herein-described and/or herein-identified nutraceutical compositions of the present invention can also include or contain Fucoidan. It is submitted that Fucoidan, a type of dietary fiber found in seaweed, is and for having immune-supportive properties, anti-inflammatory properties, and properties which support and/or repair the endocalyx. It is submitted that Fucoidan can be beneficially taken or ingested, in or with a nutraceutical composition(s) of the present invention, in the morning or at noon or at midday so as to help with absorption and to support immune function during and throughout the day.

Any of the herein-described and/or herein-identified nutraceutical compositions of the present invention can also include or contain a combination of supplements. Any of the herein-described and/or herein-identified nutraceutical compositions of the present invention can also include or contain Pterostilbene and Resveratrol. It is submitted that Pterostilbene and Resveratrol can be taken or ingested together as it is submitted that they both support heart health and longevity. It is submitted that taking or ingesting Pterostilbene and Resveratrol in a nutraceutical composition(s) of the present invention and/or with a same meal could provide, and/or enhance, benefits which are or can be associated with their combined use.

Any of the herein-described and/or herein-identified nutraceutical compositions of the present invention can also include or contain a combination of Fucoidan and/or with Vitamin C or other antioxidants. It is submitted that, in order to enhance immune function, Fucoidan can be paired with Vitamin C or other antioxidants in a nutraceutical composition(s) of the present invention in order to provide for increased antioxidant absorption and to bolster immune health.

The morning, noon or midday, and evening, nutraceutical compositions of the present invention can also include alternate nutraceutical compositions, wherein each of the respective alternate nutraceutical compositions can contain or include one of more of the following respective nutraceutical ingredients:

### I. Alternate Preferred Embodiment Morning Composition:

An alternate morning nutraceutical composition can contain or include any one or more of, or any combination of, the following:
1) NMN (Nicotinamide Mononucleotide); 2) Spermidine; 3) Rhodiola Rosea; 4) Cacao; 5) L-Theanine; 6) Glucosamine; 7) Hyaluronic Acid; and 8) Caffeine.

### II. Alternate Preferred Embodiment Noon Or Midday Composition:

An alternate noon or midday nutraceutical composition can contain or include any one or more of, or any combination of, the following:
1) EGCG (Epigallocatechin Gallate); 2) Quercetin; 3) Resveratrol; 4) Sulforaphane; 5) Calcium Alpha-Ketoglutarate; 6) Fucoidan; 7) Pterostilbene; 8) Turmeric; 9) Black Pepper/Piperine; and 10) Ginger.

### III. Alternate Preferred Embodiment Evening Composition:

An alternate evening nutraceutical composition can contain or include any one or more of, or any combination of, the following:
1) Microdose Lithium; 2) Trimethylglycine (TMG); 3) Glutathione; 4) Fisetin; 5) Urolithin A; 6) Sodium Butyrate; 7) Berberine; 8) Apigenin; 9) Cinnamon; 10) Malate; 11) Turmeric; and 12) EGCG (Epigallocatechin Gallate).

Any of the herein-described nutraceutical compositions of the present invention, which are formulated to be, and/or to be used as, a morning nutraceutical composition(s), a noon or midday nutraceutical composition(s), or an evening nutraceutical composition(s), of the present invention can be manufactured in any appropriate manner by mixing the respective ingredients of the same together and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, protein drinks, and/or any other edible food or beverages which can contain supplements such as those of, and/or provided by, the present invention.

Any and/or all of the nutraceutical compositions of the present invention described herein can also be manufactured by combining, blending, or mixing, together the respective nutraceutical ingredients of the respective nutraceutical composition by mixing and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, and/or any other edible food or beverage which can contain supplements such as those of, and/or provided by, the present invention.

Any and/or all of the respective ingredients of the respective nutraceutical composition, and/or any other suitable or appropriate nutraceutical compounds or nutraceutical ingredients, can be mixed with water, or with any suitable juice or drink, in order to obtain a nutraceutical-base product for providing longevity.

Any and/or all of the respective ingredients of the respective nutraceutical composition, and/or any other suitable or appropriate nutraceutical compounds or nutraceutical ingredients, can be mixed with green tea in order to obtain a nutraceutical-based green tea product for providing longevity.

Any and/or all of the respective ingredients of the respective nutraceutical composition, and/or any other suitable or appropriate nutraceutical compounds or nutraceutical ingredients, can be mixed with black tea in order to obtain a nutraceutical-based black tea product for providing longevity.

Any and/or all of the respective ingredients of the respective nutraceutical composition, and/or any other suitable or appropriate nutraceutical compounds or nutraceutical ingredients, can be mixed with any type or kind of tea in order to obtain a nutraceutical-based tea product for providing longevity.

Any and/or all of the respective ingredients of the respective nutraceutical composition, and/or any other suitable or appropriate nutraceutical compounds or nutraceutical ingredients, can be mixed with any type or kind of coffee, which can be caffeinated coffee, de-caffeinated coffee, or any flavored coffee, in order to obtain a nutraceutical-based coffee product for providing longevity.

Any and/or all of the respective ingredients of the respective nutraceutical composition, and/or any other suitable or appropriate nutraceutical compounds or nutraceutical ingredients, can be mixed with any type or kind of red wine in order to obtain a nutraceutical-based red wine beverage or drink product for providing longevity.

Any and/or all of the respective ingredients of the respective nutraceutical composition, and/or any other suitable or appropriate nutraceutical compounds or nutraceutical ingredients, can be mixed with any type or kind of diet beverage or drink, nutrition beverage or drink, energy beverage or drink, protein beverage or drink, or any other type or kind of beverage or drink, in order to obtain a nutraceutical-based beverage or drink product for providing longevity.

Any and/or all of the respective ingredients of the respective nutraceutical composition, and/or any other suitable or appropriate nutraceutical compounds or nutraceutical ingredients, can be mixed together in powder form and/or in liquid form, and/or in any combination of the same, and the resulting mixture can be used in producing a chewable food product for providing longevity.

Any and/or all of the nutraceutical ingredients for any respective nutraceutical composition, and/or any suitable or appropriate nutraceutical compounds or nutraceutical ingredients, can also be mixed together in powder form and/or in liquid form, and/or in any combination of the same, and the resulting mixture can be used in producing a food product such as food bar, a snack bar, a cookie, a syrup, a drink or beverage additive or supplement, or a food additive or food supplement which can be in liquid form or in powdered form, for providing longevity.

Any of the respective nutraceutical ingredients of any respective nutraceutical composition of the present invention can be mixed together by mixing and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, and/or any other edible food or beverage which can contain supplements such as those of, and/or provided by, the present invention. The resulting respective nutraceutical composition can be in liquid form or in powdered form. If a powdered form is desired from a resulting liquid nutraceutical composition, an appropriate de-hydration process or operation can be performed in the container or containment system and/or in a second or other container or containment system.

Thereafter, the resulting nutraceutical composition can be in liquid form or in powdered form can be utilized in any appropriate manner, such as in producing a longevity nutraceutical-based beverage, a longevity nutraceutical-based drink, a longevity nutraceutical-based chewable food product, a longevity nutraceutical-based food product such as food bar, a snack bar, a cookie, or a syrup, a longevity nutraceutical-based drink or beverage additive or supplement, or a longevity nutraceutical-based food additive or food supplement.

Any and/or all of the various nutraceutical compositions and/or any of the herein-described nutraceutical compositions of the present invention, which are formulated to be, and/or to be used as, a morning nutraceutical composition(s), as a noon or midday nutraceutical composition(s), or as an evening nutraceutical composition(s), of the present invention can be manufactured by, or produced by utilizing, any of the steps and/or methods described herein and/or by using any other suitable or appropriate step(s), process(es) or method(s).

The nutraceutical composition of the present invention, as well as the various nutraceutical compositions which can be formulated to be taken at different times of the day, including, but not limited to, in the morning, at noon or at midday, or in the evening, or otherwise, can also be produced so as to include any types or kinds of ingredients which are, or which can typically be, found in energy drinks, protein drinks, vitamin drinks, energy foods, protein foods, vitamin-enriched foods, energy food and/or beverage supplements, protein food and/or beverage supplements, and/or vitamin food and/or beverage supplements.

### BRIEF DESCRIPTION OF THE DRAWINGS

In the Drawings:

FIG. 1 illustrates a preferred embodiment method for producing the nutraceutical composition of the present invention and any food or beverage products and/or food or beverage supplements containing the same; and

FIG. 2 illustrates another preferred embodiment method for producing a nutraceutical composition of the present invention, using a reduced number of nutraceutical ingredients, and any food or beverage products and/or food or beverage supplements containing the same;

FIG. 3 illustrates another preferred embodiment method for producing a nutraceutical composition of the present invention in a powder form or powdered form; and

FIG. 4 illustrates another preferred embodiment method for producing a nutraceutical composition of the present invention in a liquid form or in a syrup form.

### DESCRIPTION OF THE PREFERRED EMBODIMENTS

The present invention pertains to a nutraceutical composition for combating aging in human and non-human animals and beverages, and/or food products, and/or food supplements containing the same, and, in particular, the present invention pertains to a nutraceutical composition for combating aging in human and non-human animals and beverages, and/or food products, and/or food supplements containing the same, which contain a combination of nutraceutical substances and/or products which address, and/or which subserve and/or which can serve to promote and/or facilitate the combating of, each one of twelve identified pillars of aging.

Applicant hereby incorporates by reference herein the subject matter and teachings of U.S. Provisional Patent Application Ser. No. 63/658,824, filed Jun. 11, 2024, and entitled “NUTRACEUTICAL COMPOSITION FOR COMBATING AGING IN HUMAN AND NON-HUMAN ANIMALS AND BEVERAGES AND/OR FOOD PRODUCTS AND/OR FOOD SUPPLEMENTS CONTAINING THE SAME”, the subject matter and teachings of which are hereby incorporated by reference herein in their entirety.

In a preferred embodiment, the present invention is, and provides, a nutraceutical composition which includes, but which is not limited to, at least the following 17 nutraceutical ingredients (referred to hereinafter as the “17 core nutraceutical ingredients”):
1. Berberine;
2. Trimethylglycine (TMG);
3. Microdose Lithium;
4. Epigallocatechin-3-gallate (EGCG)
5. Quercetin;
6. Fisetin;
7. Turmeric/Curcumin;
8. Resveratrol;
9. Glutathione;
10. Nicotinamide Mononucleotide (NMN) or Nicotinamide Riboside (NR);
11. Calcium Alphaketoglutarate (CaAKG);
12. Spermidine;
13. Urolithin A;
14. Sulforaphane;
15. Glucosamine;
16. Hyaluronic acid; and
17. Sodium Butyrate.

It is submitted that some of the foregoing 17 core nutraceutical ingredients may yield enhanced absorption in a liposomal preparation such as, for example, glutathione. It is also submitted that some of the 17 core nutraceutical ingredients, along with other nutraceutical ingredients, demonstrate enhanced absorption with co-administration of other compounds such as, for example, curcumin with black pepper and sulforaphane with myrosinase.

It also submitted that many of the nutraceutical ingredients noted above and further listed hereinafter are known to expand lifespan in certain organisms.

In a preferred embodiment, at least two or more of the each of the above listed 17 core nutraceutical ingredients can address and combat each of the twelve pillars of aging. In this regard, the present invention and the 17 core nutraceutical ingredients listed herein can ensure appropriate coverage for addressing and combating each of the twelve pillars of aging.

In a preferred embodiment, any other suitable nutraceutical compounds or nutraceutical ingredients can also be utilized in the present invention in addition to the 17 core nutraceutical ingredients listed above. These additional nutraceutical compounds or nutraceutical ingredients can include, but are not limited to: 1) Pterostilbene; 2) Rhodiola Rosea; 3) Malate (or Malic Acid); 4) L-Theanine; 5) Ginger; 6) Cinnamon; and/or 7) Apigenin.

In a preferred embodiment, the nutraceutical composition of the present invention can be manufactured by mixing the above-identified 17 core nutraceutical ingredients, together and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, protein drinks, and/or any other edible food or beverages which can contain supplements such as those of, and/or provided by, the present invention.

In another preferred embodiment, the nutraceutical composition of the present invention can also be manufactured by combining, blending, or mixing, together the above-identified 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, by mixing and/or by processing the processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, and/or any other edible food or beverage which can contain supplements such as those of, and/or provided by, the present invention.

In another preferred embodiment, for example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with water, or with any suitable juice or drink, in order to obtain a nutraceutical-base product for providing longevity.

In another preferred embodiment, for example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with green tea in order to obtain a nutraceutical-based green tea product for providing longevity.

In another preferred embodiment, for example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with black tea in order to obtain a nutraceutical-based black tea product for providing longevity.

In another preferred embodiment, for example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with any type or kind of tea in order to obtain a nutraceutical-based tea product for providing longevity.

In another preferred embodiment, for example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with any type or kind of coffee, which can be caffeinated coffee, de-caffeinated coffee, or any flavored coffee, in order to obtain a nutraceutical-based coffee product for providing longevity.

In another preferred embodiment, for example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with any type or kind of red wine in order to obtain a nutraceutical-based red wine beverage or drink product for providing longevity.

In another preferred embodiment, for example, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed with any type or kind of diet beverage or drink, nutrition beverage or drink, energy beverage or drink, protein beverage or drink, or any other type or kind of beverage or drink, in order to obtain a nutraceutical-based beverage or drink product for providing longevity.

In another preferred embodiment, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed together in powder form and/or in liquid form, and/or in any combination of the same, and the resulting mixture can be used in producing a chewable food product for providing longevity.

In another preferred embodiment, all of the 17 core nutraceutical ingredients, and/or any one or more of the 7 herein-mentioned additional nutraceutical compounds or nutraceutical ingredients, and/or any other suitable or appropriate nutraceutical compounds or nutraceutical ingredients, can be mixed together in powder form and/or in liquid form, and/or in any combination of the same, and the resulting mixture can be used in producing a food product such as food bar, a snack bar, a cookie, a syrup, a drink or beverage additive or supplement, or food additive or food supplement which can be in liquid form or in powdered form, for providing longevity.

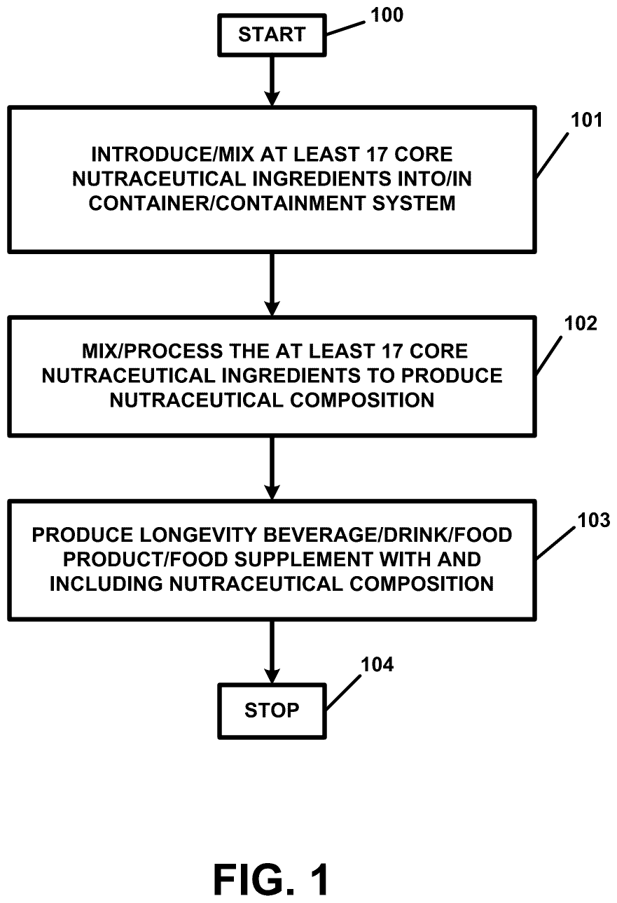

FIG. 1 illustrates a preferred embodiment method for producing the nutraceutical composition of the present invention and any food or beverage products and/or food or beverage supplements containing the same.

With reference to FIG. 1, the process for producing the nutraceutical composition of the present invention and/or the beverages, and/or food products, and/or food supplements, containing the same, commences at step 100. In a preferred embodiment, at step 101, the above-identified 17 core nutraceutical ingredients, including Berberine, Trimethylglycine, Microdose Lithium; Epigallocatechin-3-gallate (EGCG), Quercetin, Fisetin, Turmeric/Curcumin, Resveratrol, Glutathione, Nicotinamide Mononucleotide (NMN) or Nicotinamide Riboside (NR), Calcium Alphaketoglutarate, Spermidine, Urolithin A, Sulforaphane, Glucosamine, and Hyaluronic acid, each of which can be in a powdered form and/or in a liquid form, and each of which can be in respective amounts per servings as provided in Table 1, can be mixed together in any appropriate order or manner in an appropriate container or containment system.

In a preferred embodiment, at step 101, one or more of the 17 core nutraceutical ingredients can also be placed in a liposomal preparation, such as, for example, one containing glutathione. In another preferred embodiment, one or more of the 17 core nutraceutical ingredients can utilize a co-agent which can include curcumin with black pepper and/or sulforaphane with myrosinase.

TABLE 1

| Nutraceutical Ingredient | Amount (mg)/serving (range) | |
|---|---|---|
| Berberine | 500-1500 | mg |
| Trimethylglycine (TMG) | 500-2000 | mg |
| Microdose Lithium | 1-5 | mg |
| Epigallocatechin-3-gallate (EGCG) | 100-500 | mg |
| Quercetin | 250-1000 | mg |
| Fisetin | 100-1000 | mg |
| Turmeric/Curcumin | 500-2000 | mg |
| Resveratrol | 150-1000 | mg |
| Glutathione | 250-1000 | mg |
| Nicotinamide Mononucleotide (NMN) or | 250-1000 | mg |
| Nicotinamide Riboside (NR) | 100-2000 | mg |
| Calcium Alphaketoglutarate (CaAKG) | 300-2000 | mg |
| Spermidine | 0.5-10 | mg |
| Urolithin A | 250-2000 | mg |
| Sulforaphane | 1-50 | mg |
| Glucosamine | 500-3000 | mg |
| Hyaluronic acid | 50-2400 | mg |
| Sodium Butyrate | 100-3000 | mg |

In another preferred embodiment, any other or additional nutraceutical ingredient(s), such as Pterostilbene, Rhodiola Rosea, Malate (or Malic Acid), L-Theanine, Ginger; Cinnamon, and/or Apigenin, and/or any other suitable nutraceutical ingredient(s), can also be added to the mixture at step 101.

At step 102, the nutraceutical ingredients which are utilized at step 101 can be mixed together by mixing and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, and/or any other edible food or beverage which can contain supplements such as those of, and/or provided by, the present invention.

At step 102, the nutraceutical ingredients which are utilized at step 101 can be mixed together so as to yield the resulting nutraceutical composition of the present invention. In a preferred embodiment, the resulting nutraceutical composition can be in liquid form or in powdered form. In a preferred embodiment, at step 102, if a powdered form is desired from a resulting liquid nutraceutical composition, an appropriate de-hydration process or operation can be performed in the container or containment system and/or in a second or other container or containment system.

Thereafter, the resulting nutraceutical composition can be in liquid form or in powdered form can be utilized in any appropriate manner, such as in producing a longevity nutraceutical-based beverage, a longevity nutraceutical-based drink, a longevity nutraceutical-based chewable food product, a longevity nutraceutical-based food product such as food bar, a snack bar, a cookie, or a syrup, a longevity nutraceutical-based drink or beverage additive or supplement, or a longevity nutraceutical-based food additive or food supplement.

At step 103, the respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement, can be produced using the nutraceutical composition (also referred to as the “resulting nutraceutical composition”) which was produced at and/or during step 102. In a preferred embodiment, the respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement, can be produced using any respective, suitable, and/or appropriate method(s), technique(s), process(es), and/or operation(s) known by those skilled in the art of food production or manufacturing, beverage or drink production or manufacturing, food supplement production or manufacturing, and/or beverage or drink supplement production or manufacturing.

In a preferred embodiment, at or during step 103, any natural flavoring(s) and/or any artificial flavoring(s) can be added to the respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement.

Thereafter, and once the respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement is produced, the method the preferred embodiment will cease at step 104.

In another preferred embodiment, the present invention is, and provides, a nutraceutical composition which can include a reduced number of nutraceutical ingredients while still providing for the addressing and the combating of all twelve of the pillars of aging. In one such preferred embodiment, twelve or more nutraceutical ingredients can be utilized which can be selected from among the following 24 nutraceutical ingredients:
1. Berberine;
2. Trimethylglycine (TMG);
3. Microdose Lithium;
4. Epigallocatechin-3-gallate (EGCG)
5. Quercetin;
6. Fisetin;
7. Turmeric/Curcumin;
8. Resveratrol;
9. Glutathione;
10. Nicotinamide Mononucleotide (NMN) or Nicotinamide Riboside (NR);
11. Calcium Alphaketoglutarate (CaAKG);
12. Spermidine;
13. Urolithin A;
14. Sulforaphane;
15. Glucosamine;
16. Hyaluronic acid;
17) Sodium Butyrate;
18) Pterostilbene;
19) Rhodiola Rosea;
20) Malate (or Malic Acid);
21) L-Theanine;
22) Ginger;
23) Cinnamon; and
24) Apigenin.

Table 2 lists the above 24 nutraceutical ingredients along with suggested dosages per serving of nutraceutical composition.

TABLE 2

| Nutraceutical Ingredient | Amount (mg)/serving (range) | |
|---|---|---|
| Berberine | 500-1500 | mg |
| Trimethylglycine (TMG) | 500-2000 | mg |
| Microdose Lithium | 1-5 | mg |
| Epigallocatechin-3-gallate (EGCG) | 100-500 | mg |
| Quercetin | 250-1000 | mg |
| Fisetin | 100-1000 | mg |
| Turmeric/Curcumin | 500-2000 | mg |
| Resveratrol | 150-1000 | mg |
| Glutathione | 250-1000 | mg |
| Nicotinamide Mononucleotide (NMN) or | 250-1000 | mg |
| Nicotinamide Riboside (NR) | 100-2000 | mg |
| Calcium Alphaketoglutarate (CaAKG) | 300-2000 | mg |
| Spermidine | 0.5-10 | mg |
| Urolithin A | 250-2000 | mg |
| Sulforaphane | 1-50 | mg |
| Glucosamine | 500-3000 | mg |
| Hyaluronic acid | 100-2400 | mg |
| Ginger | 500-3000 | mg |
| L-Theanine | 50-200 | mg |
| Malate | 1000-6000 | mg |
| Rhodiola Rosea | 200-600 | mg |
| Pterostilbene | 50-250 | mg |
| Apigenin | 50-500 | mg |
| Cinnamon | 1000-6000 | mg |
| Sodium Butyrate | 100-3000 | mg |

In another preferred embodiment, less than thirteen of the foregoing 24 nutraceutical ingredients, or any other nutraceutical ingredients not identified herein, can also be utilized provided the nutraceutical composition formed thereby can effectively address and combat all twelve of the pillars of aging in an effective manner.

In another preferred embodiment, at least the following thirteen nutraceutical ingredients can be utilized in producing the nutraceutical composition of the present invention:
1. Berberine;
2. Trimethylglycine (TMG);
3. Microdose Lithium;
4. Epigallocatechin-3-gallate (EGCG)
5. Fisetin;
6. Resveratrol;
7. Glutathione;
8. Nicotinamide Mononucleotide (NMN) or Nicotinamide Riboside (NR);
9. Calcium Alphaketoglutarate (CaAKG);
10. Spermidine;
11. Urolithin A;
12. Hyaluronic acid; and
13. Sodium Butyrate

In another preferred embodiment, one or more of any other nutraceutical ingredients identified herein, or any other appropriate and/or suitable nutraceutical ingredient(s) not described herein, can also be utilized along with the above 13 listed nutraceutical ingredients.

In a preferred embodiment, at least the above listed 13 nutraceutical ingredients, including: Berberine, Trimethylglycine (TMG), Microdose Lithium, Epigallocatechin-3-gallate (EGCG), Fisetin, Resveratrol, Glutathione, Nicotinamide Mononucleotide (NMN) or Nicotinamide Riboside (NR), Calcium Alphaketoglutarate (CaAKG), Spermidine, Urolithin A, and Hyaluronic acid, can be utilized to provide for a minimal or a reduced number of nutraceutical ingredients which can effectively address and combat all twelve of the pillars of aging.

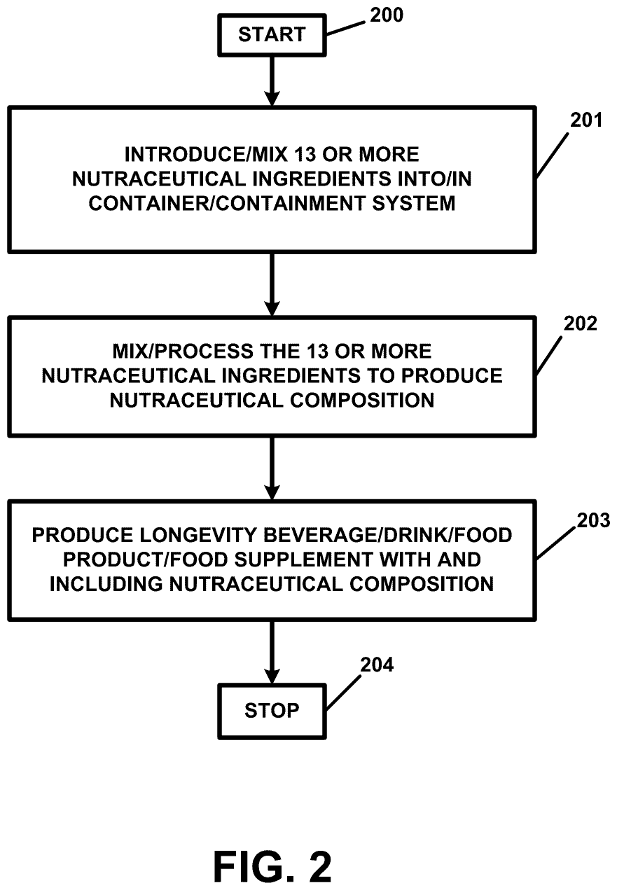

FIG. 2 illustrates another preferred embodiment method for producing a nutraceutical composition of the present invention, using a reduced number (thirteen or more) of nutraceutical ingredients, and any food or beverage products and/or food or beverage supplements containing the same.

With reference to FIG. 2, the process for producing the nutraceutical composition of the present invention and/or the beverages, and/or food products, and/or food supplements, containing the same, commences at step 200. In a preferred embodiment, at step 201, the above-identified 13 nutraceutical ingredients, including Berberine, Trimethylglycine, Microdose Lithium; Epigallocatechin-3-gallate (EGCG), Fisetin, Resveratrol, Glutathione, Nicotinamide Mononucleotide (NMN) or Nicotinamide Riboside (NR), Calcium Alphaketoglutarate, Spermidine, Urolithin A, and Hyaluronic acid, each of which can be in a powdered form and/or in a liquid form, and each of which can be in respective amounts per servings as provided in Table 1 or 2, can be mixed together in any appropriate order or manner in an appropriate container or containment system.

In a preferred embodiment, at step 201, one or more of the 13 nutraceutical ingredients can also be placed in a liposomal preparation, such as, for example, one containing glutathione. In another preferred embodiment, one or more of the 13 core nutraceutical ingredients can utilize a co-agent which can include curcumin with black pepper (which contains Piperine) and/or sulforaphane with myrosinase.

In another preferred embodiment, any other or additional nutraceutical ingredient(s), such as Pterostilbene, Rhodiola Rosea, Malate (or Malic Acid), L-Theanine, Ginger; Cinnamon, and/or Apigenin, and/or any other suitable nutraceutical ingredient(s) from Table 1 or Table 2, can also be added to the mixture at step 201.

At step 202, the nutraceutical ingredients which are utilized at step 201 can be mixed together by mixing and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, and/or any other edible food or beverage which can contain supplements such as those of, and/or provided by, the present invention.

At step 202, the nutraceutical ingredients which are utilized at step 201 can be mixed together so as to yield the resulting nutraceutical composition of the present invention. In a preferred embodiment, the resulting nutraceutical composition can be in liquid form or in powdered form. In a preferred embodiment, at step 202, if a powdered form is desired from a resulting liquid nutraceutical composition, an appropriate de-hydration process or operation can be performed in the container or containment system and/or in a second or other container or containment system.

Thereafter, the resulting nutraceutical composition can be in liquid form or in powdered form can be utilized in any appropriate manner, such as in producing a longevity nutraceutical-based beverage, a longevity nutraceutical-based drink, a longevity nutraceutical-based chewable food product, a longevity nutraceutical-based food product such as food bar, a snack bar, a cookie, or a syrup, a longevity nutraceutical-based drink or beverage additive or supplement, or a longevity nutraceutical-based food additive or food supplement.

At step 203, the respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement, can be produced using the nutraceutical composition (also referred to as the “resulting nutraceutical composition”) which was produced at and/or during step 202. In a preferred embodiment, the respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement, can be produced using any respective, suitable, and/or appropriate method(s), technique(s), process(es), and/or operation(s) known by those skilled in the art of food production or manufacturing, beverage or drink production or manufacturing, food supplement production or manufacturing, and/or beverage or drink supplement production or manufacturing.

In a preferred embodiment, at or during step 203, any natural flavoring(s) and/or any artificial flavoring(s) can be added to the respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement.

Thereafter, and once the respective longevity nutraceutical-based beverage, longevity nutraceutical-based drink, longevity nutraceutical-based chewable food product, longevity nutraceutical-based food product such as food bar, snack bar, cookie, brownie, or syrup, longevity nutraceutical-based drink or beverage additive or supplement, or longevity nutraceutical-based food additive or food supplement is produced, the method the preferred embodiment will cease at step 204.

As noted above, in another preferred embodiment, the nutraceutical composition of the present invention can also be produced from less than thirteen nutraceutical ingredients, with the minimum number only being limited depending upon the addressing and effective combating of all twelve of the pillars of aging.

The nutraceutical composition of the present invention can be used to provide a pre-packaged beverage or drink, a pre-packaged tea beverage or drink, a pre-packaged coffee beverage or drink, a pre-packaged red wine beverage or drink, a pre-packaged carbonated beverage or drink, a pre-packaged caffeinated beverage or drink, a product which can be offered in powdered form, a chewable food product, a food product, a food snack bar product, a food supplement product which can be provided in liquid form or in powdered form, and/or any other consumable beverage, drink, edible product, food product, or food supplement. In a preferred embodiment, the present invention can provide a nutraceutical composition which can be consumed in any beverage, drink, edible, food product, or food supplement, daily by itself as a stand alone supplement and/or as a daily solution to address and combat the problem of aging while dispensing with the need to obtain and mix the individual ingredients prior to consumption.

In another preferred embodiment, the nutraceutical composition of the present invention can also contain the nutraceutical Fucoidan in a range of 100-4000 mg. In a preferred embodiment, the Fucoidan can be an additional ingredient in any nutraceutical composition of, and prepared pursuant to, the present invention. In another preferred embodiment, Fucoidan can be used as a suitable substitute for any ingredient in any nutraceutical composition of, and prepared pursuant to, the present invention.

In another preferred embodiment, the nutraceutical composition of the present invention can also contain the nutraceutical Cacao in any suitable and safe amount or range provided that said amount does not exceed any safe or recommended daily dosage amount or range. In a preferred embodiment, the Cacao can be an additional ingredient in any nutraceutical composition of, and prepared pursuant to, the present invention. In another preferred embodiment, Cacao can be used as a suitable substitute for any ingredient in any nutraceutical composition of, and prepared pursuant to, the present invention.

In another preferred embodiment, the nutraceutical composition of the present invention can be manufactured and sold in powder form as a powder which can be added to, and/or mixed with, any drink or beverage, or which can be added to, and/or mixed with, any food. In a preferred embodiment, the nutraceutical composition of the present invention, which can be manufactured as a powder, can be flavored with any appropriate and/or suitable flavor or flavoring, which flavor or flavoring can be, or can include, a natural flavor or flavoring or any combination of natural flavors or flavorings.

In another preferred embodiment, the nutraceutical composition of the present invention, which can be manufactured as a powder, can be flavored with any appropriate and/or suitable flavor or flavoring, which flavor or flavoring can be, or can include, an artificial flavor or flavoring or any combination of artificial flavors or flavorings.

In another preferred embodiment, the nutraceutical composition of the present invention, which can be manufactured as a powder, can be flavored with any appropriate and/or suitable flavor or flavoring, which flavor or flavoring can be, or can include, any combination of a natural flavor or flavoring and an artificial flavor or flavoring or any combination of an artificial flavor or flavoring and one or more natural flavor(s) or flavoring(s) or any combination of a natural flavor or flavoring and one or more artificial flavor(s) or flavoring(s).

In another preferred embodiment, the nutraceutical composition of the present invention, which can be manufactured as a powder, can also be non-flavored.

In a preferred embodiment, the nutraceutical composition of the present invention, which can be manufactured as a powder, can also produced with caffeine as a caffeinated product. In another preferred, the nutraceutical composition of the present invention, which can be manufactured as a powder, can also produced with without caffeine as a non-caffeinated product or as a de-caffeinated product.

In another preferred embodiment, nutraceutical composition of the present invention can also be produced or manufactured as a concentrated liquid or as a syrup which can be added to, and/or mixed with, any drink or beverage, or which can be added to, and/or mixed with, any food. In a preferred embodiment, the nutraceutical composition of the present invention, which can also be produced as a concentrated liquid or as a syrup, can be flavored with any natural flavor or flavoring, or can be flavored with any artificial flavor or flavoring, or can be flavored with any combination of one or more natural flavor(s) or flavoring(s) and one or artificial flavor(s) or flavoring(s).

In another preferred embodiment, the nutraceutical composition of the present invention, which can also be produced as a concentrated liquid or as a syrup, can be unflavored. In another preferred embodiment, the nutraceutical composition of the present invention, which can also be produced as a concentrated liquid or as a syrup, can be caffeinated or can be uncaffeinated or de-caffeinated.

In another preferred embodiment, the herein-identified nutraceutical ingredients, including, but not limited to, Berberine, Trimethylglycine (TMG), Microdose Lithium, Epigallocatechin-3-gallate (EGCG), Fisetin, Resveratrol, Glutathione, Nicotinamide Mononucleotide (NMN) or Nicotinamide Riboside (NR), Calcium Alphaketoglutarate (CaAKG), Spermidine, Urolithin A, and Hyaluronic acid, or any combination of any of the herein-identified nutraceutical ingredients, which can include any of the ingredients identified in Table 2 and/or Fucoidan and/or cacao, can also be utilized to provide for a nutraceutical composition which can effectively address and combat all twelve of the pillars of aging.

In another preferred embodiment, the nutraceutical composition of the present invention can be mixed and/or processed in order to yield a nutraceutical composition in a powder form or powdered form which can be added to, or mixed into and/or with, water or any suitable liquid drink or beverage, or a juice, coffee, tea, or any other type or kind of beverage. In a preferred embodiment, the nutraceutical composition of the present invention can be mixed and/or processed in order to yield a nutraceutical composition in a powder form or powdered form which can be added to, or mixed into and/or with, any cold liquid or beverage or any hot or warm liquid of beverage.

In a preferred embodiment, for example, the nutraceutical composition in powder form or powdered form can be packaged in a stick package, in a stick packet, or/and in any suitable packet or packaging. In a preferred embodiment, such a packaged nutraceutical composition provided in powder form can be mixed into or with any water or other liquid or beverage.

In another preferred embodiment, the nutraceutical composition of the present invention can be mixed and/or processed in order to yield a nutraceutical composition in a liquid form or in a syrup form, which nutraceutical composition can be added to, or mixed into and/or with, water or any suitable liquid drink or beverage, or a juice, coffee, tea, or any other type or kind of beverage.

In another preferred embodiment, the nutraceutical composition of the present invention, in a liquid form or in a syrup form, can be added to, or mixed into and/or with, any cold liquid or beverage or any hot or warm liquid of beverage. In a preferred embodiment, for example, the nutraceutical composition in liquid form or in syrup form can be packaged in any suitable container, bottle, or can, made of any suitable material. In a preferred embodiment, such a packaged nutraceutical composition, in liquid form or in syrup form can be mixed into or with any water or other liquid or beverage.

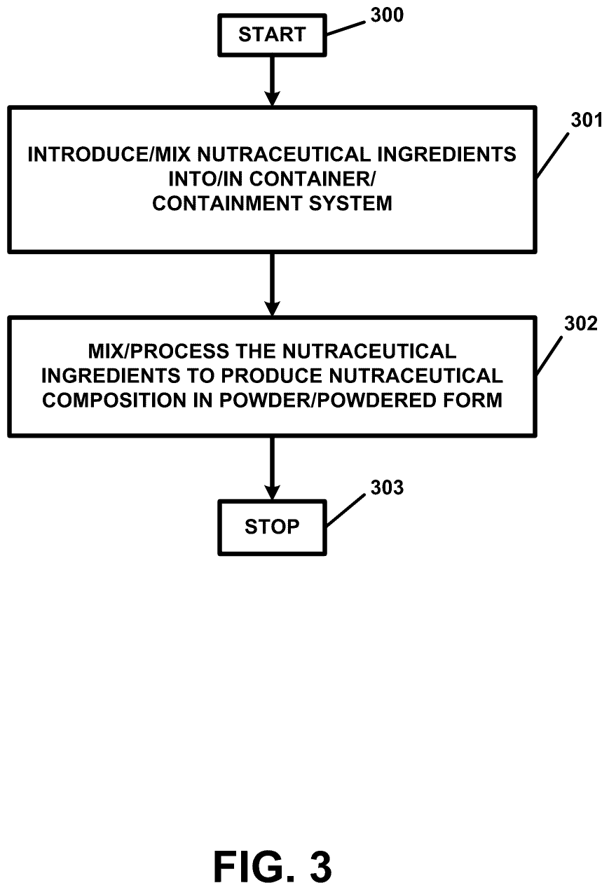

FIG. 3 illustrates another preferred embodiment method for producing the nutraceutical composition of the present invention. With reference to FIG. 3, the process for producing the nutraceutical composition of the present invention in a powder form or powdered form commences at step 300.

In a preferred embodiment, at step 301, the nutraceutical ingredients, including, but not limited to, Berberine, Trimethylglycine, Microdose Lithium; Epigallocatechin-3-gallate (EGCG), Fisetin, Resveratrol, Glutathione, Nicotinamide Mononucleotide (NMN) or Nicotinamide Riboside (NR), Calcium Alphaketoglutarate, Spermidine, Urolithin A, and Hyaluronic acid, and/or any of other ingredients identified in Table 2, and/or Fucoidan, and/or cacao, each of which can be in their respective amounts per servings as provided in Table 1 or Table 2, or as provided elsewhere in this patent application, and/or as desired, can be mixed together in any appropriate order or manner in an appropriate container or containment system.

In a preferred embodiment, at step 301, one or more of the nutraceutical ingredients can also be placed in a liposomal preparation, such as, for example, one containing glutathione. In another preferred embodiment, one or more of the nutraceutical ingredients can also utilize a co-agent which can include curcumin with black pepper (which contains Piperine) and/or sulforaphane with myrosinase.

At step 302, the nutraceutical ingredients which are utilized at step 301 can be mixed together by mixing and/or by processing the same in any, and/or during any, appropriate mixing process or operation, which are known to those having skill in the art of the preparation of powder compositions or powdered compositions and/or mixtures. In another preferred embodiment, any natural flavor or flavoring, and/or any artificial flavor or flavoring, can also be added to the mixture of the nutraceutical ingredients at step 302. In another preferred embodiment, caffeine can also be added to the mixture of the nutraceutical ingredients at step 302.

At step 302, the nutraceutical ingredients, which are utilized at step 301, can be mixed together and/or processed so as to yield the resulting nutraceutical composition of the present invention in a powder form or powdered form.

Thereafter, once the nutraceutical composition of the present invention in a powder form or powdered form is prepared, the method the preferred embodiment of FIG. 3 will cease at step 303.

When it is desired to add or mix the nutraceutical composition, in a powder form or powdered form, with any desired water, liquid, or beverage, the same can simply be added to, and/or mixed into or with, the desired water, liquid, or beverage.

In another preferred embodiment, the nutraceutical composition of the present invention can be prepared in liquid form or in syrup form.

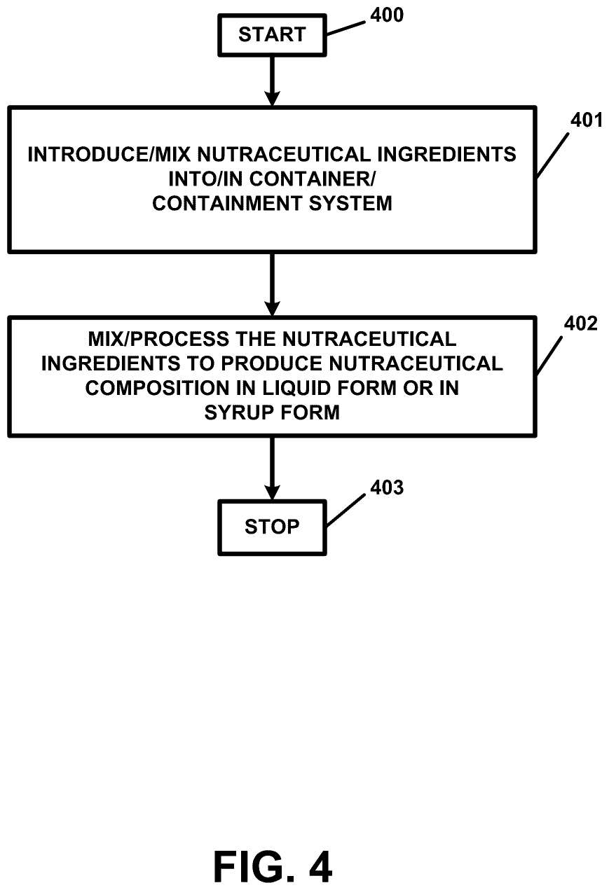

FIG. 4 illustrates another preferred embodiment method for producing the nutraceutical composition of the present invention in a liquid form or in a syrup form. With reference to FIG. 4, the process for producing the nutraceutical composition of the present invention in a liquid form or in a syrup form commences at step 400.

In a preferred embodiment, at step 401, the nutraceutical ingredients, including, but not limited to, any combination of those including Berberine, Trimethylglycine, Microdose Lithium; Epigallocatechin-3-gallate (EGCG), Fisetin, Resveratrol, Glutathione, Nicotinamide Mononucleotide (NMN) or Nicotinamide Riboside (NR), Calcium Alphaketoglutarate, Spermidine, Urolithin A, and Hyaluronic acid, and/or any of other ingredients identified in Table 2, and/or Fucoidan, and/or cacao, each of which can be in their respective amounts per servings as provided in Table 1 or Table 2, or as provided elsewhere in this patent application, and/or as desired, can be mixed together in any appropriate order or manner in an appropriate container or containment system.

In a preferred embodiment, at step 401, one or more of the nutraceutical ingredients can also be placed in a liposomal preparation, such as, for example, one containing glutathione. In another preferred embodiment, one or more of the nutraceutical ingredients can also utilize a co-agent which can include curcumin with black pepper (which contains Piperine) and/or sulforaphane with myrosinase.

At step 402, the nutraceutical ingredients which are utilized at step 401 can be mixed together by mixing and/or by processing the same in any, and/or during any, appropriate mixing process or operation, which are known to those having skill in the art of the preparation of a liquid product or liquid form product or a syrup product or a syrup form product. In another preferred embodiment, any natural flavor(s) or flavoring(s), and/or any artificial flavor(s) or flavoring(s), can also be added to the mixture of the nutraceutical ingredients at step 402. In another preferred embodiment, caffeine can also be added to the mixture of the nutraceutical ingredients at step 402.

At step 402, the nutraceutical ingredients which are utilized at step 401 can be mixed together and/or processed so as to yield the resulting nutraceutical composition of the present invention in a liquid form or in a syrup form.

Thereafter, once the nutraceutical composition of the present invention in a liquid form or in a syrup form is prepared, the method the preferred embodiment of FIG. 4 will cease at step 403.

When it is desired to add or mix the nutraceutical composition, in a liquid for or in a syrup form, with any desired water, liquid, or beverage, the same can simply be added to, and/or mixed into or with, the desired water, liquid, or beverage.

In a preferred embodiment, the present invention can provide a food product containing the nutraceutical composition which can be a food supplement which can be added to a food, a beverage, or a drink.

In a preferred embodiment, the present invention can provide a food product containing the nutraceutical composition which can be a food supplement which can be applied to a food, a beverage, or a drink. In a preferred embodiment, the food product or food supplement of the present invention can also be sprinkled or poured on or over, or into, any food, beverage, or drink.

It is submitted that the nutraceutical composition of the present invention and any products containing the same can provide benefits in and to all major organ systems. These benefits could include, but are not limited to, improved metabolic health, improved cardiovascular health, decreased inflammation, enhanced anti-oxidant benefits, cancer prevention, improved gastrointestinal health, neuroprotection, mood stabilization, improved cognitive health, improved brain health, improved and enhanced antibacterial and anti-viral support, improved and enhanced immune system support, improved respiratory health, improved and enhanced exercise performance and recovery, improved skin health, improved joint health, improved liver health, improved blood sugar control, improved weight management, improved detoxification, improved cellular health and repair, improved energy metabolism, improved protein synthesis, improved wound healing, improved cellular health, improved mitochondrial health, and improved and enhanced life span extension.

The nutraceutical composition of the present invention serves to address and combat the twelve pillars of aging. Further, the nutraceutical composition of the present invention serves to address and combat the twelve pillars of aging in an efficient and effective manner.

Applicant hereby incorporates by reference herein all processes, proprietary and non-proprietary, which are known to those skilled in the pertinent and respective art(s), as of the date of the filing of this patent application, in order to perform any of the method(s), technique(s), process(es), and/or operation(s), needed or desired in performing any of the food production or manufacturing, the beverage or drink production or manufacturing, the food supplement production or manufacturing, and/or the beverage or drink supplement production or manufacturing, in producing any beverages, drinks, foods, and/or food supplements, described herein as being produced using any of the nutraceutical compositions of the present invention.

Applicant also hereby incorporates by reference herein the subject matter and teachings of U.S. Pat. No. 11,839,622, entitled “CONSUMABLE NUTRACEUTICAL COMPOSITION”, the subject matter and teachings of which are incorporated by reference herein in their entirety, as if to be fully restated herein, so as incorporate the teachings of same, and so as to improve upon the teachings of the same.

In another preferred embodiment, as well as in any and/or all of the embodiments described herein, the nutraceutical composition of the present invention can also be produced so as to include any types or kinds of ingredients typically found in energy drinks, protein drinks, vitamin drinks, energy foods, protein foods, vitamin-enriched foods, energy food and/or beverage supplements, protein food and/or beverage supplements, and/or vitamin food and/or beverage supplements.

In another preferred embodiment, the present invention can be, and/or can include, multiple nutraceutical compositions which can each contain or include different nutraceutical ingredients, and which can be designed to be administered to, or ingested by, an individual at different times of the day.

It is submitted that various nutraceutical products or ingredients can have absorption rates, can interact differently with, and/or can impact metabolic rates, and/or can have other effects, which can differ depending upon the time of the day at which they are ingested, and that the same can differ depending upon any other nutraceutical products or ingredients which are ingested at the same time.

It is also submitted that the specific properties of each nutraceutical product or ingredient should be considered when seeking to effectuate optimal absorption and synergies when taking the nutraceutical product or ingredient along with another nutraceutical product or ingredient or with other nutraceutical products or ingredients.

In another preferred embodiment, the present invention can be, and/or can include, multiple nutraceutical compositions, each of which is, or can be, designed to be taken by, or ingested by, an individual, or administered to an individual, at different times of day in order to facilitate optimal effectiveness in use. In a preferred embodiment, each of the multiple nutraceutical compositions of the present invention can be selected and/or formulated for optimal use at its respective time of ingestion.

In a preferred embodiment, the present invention can be, and/or can include, three nutraceutical compositions, wherein the first nutraceutical composition is ingested in the morning, the second nutraceutical composition is ingested at midday, and the third nutraceutical composition is ingested in the evening. In a preferred embodiment, the nutraceutical ingredients selected for inclusion in and/or use in each of the respective first nutraceutical composition, second nutraceutical composition, and the third nutraceutical composition, can be selected for their respective properties and effects.

In another preferred embodiment, the present invention can be, and/or can include, two or more, or any number of, nutraceutical compositions, each of which can be ingested at particular and/or specified times of day in order to provide optimal effectiveness, and/or optimal absorption and/or synergies. In this regard, it is submitted that rates of absorption, time of day optimal effectiveness, and metabolic interactions between and among various nutraceutical products, can all play a factor in providing for an effective and efficient use of nutraceutical products and/or in achieving optimal results from the use of the same by individuals. In this regard, in a preferred embodiment, the present invention can include multiple nutraceutical compositions, with each being taken or ingested at particular times of the day, such as, for example, but not limited to, in the morning, at noon or at midday, or in the evening, and/or at any other time or times and/or pursuant to any suitable or appropriate schedule.

In a preferred embodiment, the present invention is or can be directed to at least three nutraceutical compositions, each of which can be taken or ingested at at least three separate times of the day, which, in a preferred embodiment, can be in the morning, at noon or at midday, or in the evening, respectively. In such a preferred embodiment, each of the respective nutraceutical compositions can include any combination of the following nutraceutical ingredients or nutraceuticals:

### I. First Nutraceutical Composition (Morning Composition):

In a preferred embodiment, a first nutraceutical composition, which can be taken or ingested in the morning, can contain or include any one or more of, or any combination of, the following:
A. NMN (Nicotinamide Mononucleotide)
B. Spermidine
C. Rhodiola Rosea
D. Turmeric
E. Ginger
F. Cacao
G. L-Theanine
H. Glucosamine
I. Hyaluronic Acid.

In a preferred embodiment, the First Nutraceutical Composition should be taken or ingested in the morning. It is submitted that NMN (Nicotinamide Mononucleotide) supports energy levels, thereby making the same beneficial for being ingested in the morning. It is submitted that Spermidine supports cellular health throughout the day, thereby making the same beneficial for being ingested in the morning. It is submitted that Rhodiola Rosea has energizing effects, thereby making the same beneficial and suitable for being ingested in the morning. It is submitted that Turmeric can be ingested in the morning or at midday with a fat-containing meal for better, or for more optimal, absorption into the body, thereby making the same beneficial and suitable for being ingested in the morning or at midday.

It is submitted that Ginger supports digestion and can be taken with breakfast, lunch, or dinner, thereby making the same beneficial and suitable for being ingested in the morning, at midday, and in the evening. It is submitted that Cacao contains Caffeine and other stimulants, which are best taken in the morning, thereby making the same beneficial and suitable for being ingested in the morning. It is submitted that L-Theanine, alone and/or taken with Caffeine, can provide a calming effect without sedation, thereby making the same beneficial and suitable for being ingested in the morning as well as in the evening. It is also submitted that L-Theanine, can also be taken as an ingredient of a nutraceutical composition which can be taken or ingested in the evening without Caffeine.

It is submitted that Glucosamine, often used for joint support, can be beneficial and suitable for being ingested in the morning. It is submitted that Hyaluronic Acid, which is useful for skin and joint health, can be beneficial and suitable for being ingested in the morning.

It is submitted that rates of absorption, time of day optimal effectiveness, and metabolic interactions between and among various nutraceutical products, can all play a factor in providing for the effective and efficient use of nutraceutical products and/or in achieving optimal results from the use of the same by individuals.

### II. Second Nutraceutical Composition (Noon or Midday Composition):

In a preferred embodiment, a second nutraceutical composition, which can be taken or ingested at noon or at midday, can contain or include any one or more of, or any combination of, the following:
A. EGCG (Epigallocatechin Gallate)
B. Quercetin
C. Resveratrol
D. Sulforaphane
E. Calcium Alpha-Ketoglutarate.

In a preferred embodiment, the Second Nutraceutical Composition should be taken or ingested at noon or at midday. It is submitted that EGCG (Epigallocatechin Gallate), which can typically be found in green tea, can be best taken or ingested at noon or at midday in order to avoid interference with iron absorption and in order to avoid sleep disturbances. It is submitted that Quercetin can best be taken or ingested with, and/or can be paired with, a noon or midday meal in order aid in its absorption. It is submitted that Resveratrol can also be taken or ingested with, and/or can be paired with, a noon or midday meal containing fat in order provide for better absorption. It is submitted that Sulforaphane, typically found in, or obtained from, cruciferous vegetable, can also be taken or ingested at noon or midday in order to help with detoxification processes. Lastly, it is submitted that Calcium Alpha-Ketoglutarate can be taken or ingested along with, and/or paired with, a meal at noon or at midday, in order to aid in or along with energy metabolism.

### III. Third Nutraceutical Composition (Evening Composition):

In a preferred embodiment, a third nutraceutical composition, which can be taken or ingested in the evening, can contain or include any one or more of, or any combination of, the following:
A. Microdose Lithium
B. Trimethylglycine (TMG)
C. Glutathione
D. Fisetin
E. Urolithin A
F. Sodium Butyrate
G. Berberine
H. Apigenin
I. Cinnamon
J. Malate.

In a preferred embodiment, the Third Nutraceutical Composition should be taken or ingested in the evening. It is submitted that Microdose Lithium is recommended for evening use due to its potential effects on mood and sleep. It is submitted that Trimethylglycine (TMG) supports liver health and can aid in overnight detoxification processes and, as such, can be beneficial if taken or ingested in the evening. It is submitted that Glutathione, which can be involved in detoxification processes, can also be taken or ingested in the evening in order to support overnight cellular repair processes. It is submitted that Fisetin is an antioxidant which can be taken in the evening in order to help with its potential senolytic effects. It is submitted that Urolithin A is linked to mitochondrial health and well being and can also be taken or ingested in the evening.

It is submitted that Sodium Butyrate, which is linked to gut health and well being and/or gastro-intestinal health and well being, and can also be taken or ingested in the evening. It is submitted that Berberine, if taken or ingested for glucose metabolism, can best be taken or ingested at a time of or near a last meal of the day or before bedtime. It is submitted that Apigenin, which can be known for its calming effects, can also be taken in the evening. It is submitted that Cinnamon, which can be known for its post-meal glucose management benefits, can also be beneficially taken or ingested after dinner or a last meal of day. Lastly, it is submitted that Malate, which can be known for its involvement in the Krebs cycle, can support energy processes during rest, can also be beneficially taken or ingested in the evening.

In addition to the foregoing, each of the above nutraceutical compositions, as well as each of any and/or all of the various nutraceutical compositions disclosed herein, can also include antioxidants, fat soluble supplements, and/or pro-energetic supplements, in order to provide other benefits. As and for an example, antioxidants, such as, but not limited to, Quercetin, Resveratrol, and Fisetin, can also be included in, and/or taken together, in a nutraceutical composition as they can support each other in combating oxidative stress. In this regard, in another preferred embodiment, any nutraceutical composition disclosed herein can also include Quercetin, Resveratrol, and Fisetin.

As and for another example, fat soluble supplements, such as, but not limited to, Turmeric and Resveratrol, should be taken or ingested with meals containing fats for better absorption. In this regard, in another preferred embodiment, any nutraceutical composition disclosed herein which can be taken or ingested with or in the vicinity of a meal containing fats can also include Turmeric and/or Resveratrol and/or any other fat-soluble supplement(s).

As and for another example, pro-energetic supplements, such as, but not limited to, Nicotinamide Mononucleotide (NMN), Spermidine, and Calcium Alphaketoglutarate (CaAKG), can generally be best taken or ingested during the first half of the day so as to be best aligned with the body's natural peak in metabolic activities. In this regard, in another preferred embodiment, any nutraceutical composition disclosed herein which can be taken or ingested in or during the first half of the day can also include Nicotinamide Mononucleotide (NMN), Spermidine, and Calcium Alphaketoglutarate (CaAKG), and/or any other pro-energetic supplement(s).

In another preferred embodiment, any of the herein-described and/or herein-identified nutraceutical compositions of the present invention can also include or contain Pterostilbene which is submitted to be a compound which is similar to Resveratrol and having antioxidant properties as well as anti-inflammatory properties. It is also submitted that, since Pterostilbene can have a potential impact on cognitive function and overall health, Pterostilbene can be taken or ingested, in or with a nutraceutical composition(s) of the present invention, in the morning or at noon or at midday so as to more efficiently and more effectively utilizing its energizing effects throughout the day as well as possible or potentially aiding in maintaining focus and/or vitality.

In another preferred embodiment, any of the herein-described and/or herein-identified nutraceutical compositions of the present invention can also include or contain Fucoidan. It is submitted that Fucoidan, a type of dietary fiber found in seaweed, is and for having immune-supportive properties, anti-inflammatory properties, and properties which support and/or repair the endocalyx. It is submitted that Fucoidan can be beneficially taken or ingested, in or with a nutraceutical composition(s) of the present invention, in the morning or at noon or at midday so as to help with absorption and to support immune function during and throughout the day.

In another preferred embodiment, any of the herein-described and/or herein-identified nutraceutical compositions of the present invention can also include or contain a combination of supplements. In a preferred embodiment, any of the herein-described and/or herein-identified nutraceutical compositions of the present invention can also include or contain Pterostilbene and Resveratrol. It is submitted that Pterostilbene and Resveratrol can be taken or ingested together as it is submitted that they both support heart health and longevity. It is submitted that taking or ingesting Pterostilbene and Resveratrol in a nutraceutical composition(s) of the present invention and/or with a same meal could provide, and/or enhance, benefits which are or can be associated with their combined use.

In another preferred embodiment, any of the herein-described and/or herein-identified nutraceutical compositions of the present invention can also include or contain a combination of Fucoidan and/or with Vitamin C or other antioxidants. It is submitted that, in order to enhance immune function, Fucoidan can be paired with Vitamin C or other antioxidants in a nutraceutical composition(s) of the present invention in order to provide for increased antioxidant absorption and to bolster immune health.

In another preferred embodiment, the morning, noon or midday, and evening, nutraceutical compositions of the present invention can include any one of more of the following respective nutraceutical ingredients:

### I. Alternate Preferred Embodiment Morning Composition:

As and for another preferred embodiment for a morning nutraceutical composition, the nutraceutical composition of the present invention can contain or include any one or more of, or any combination of, the following:
A. NMN (Nicotinamide Mononucleotide)
B. Spermidine
C. Rhodiola Rosea
D. Cacao
E. L-Theanine
F. Glucosamine
G. Hyaluronic Acid
H. Caffeine.

### II. Alternate Preferred Embodiment Noon Or Midday Composition:

As and for another preferred embodiment for a noon or midday nutraceutical composition, the nutraceutical composition of the present invention can contain or include any one or more of, or any combination of, the following:
A. EGCG (Epigallocatechin Gallate)
B. Quercetin
C. Resveratrol
D. Sulforaphane
E. Calcium Alpha-Ketoglutarate
F. Fucoidan
G. Pterostilbene
H. Turmeric
I. Black Pepper/Piperine
J. Ginger.

### III. Alternate Preferred Embodiment Evening Composition:

As and for another preferred embodiment for an evening nutraceutical composition, the nutraceutical composition of the present invention can contain or include any one or more of, or any combination of, the following:
A. Microdose Lithium
B. Trimethylglycine (TMG)
C. Glutathione
D. Fisetin
E. Urolithin A
F. Sodium Butyrate
G. Berberine
H. Apigenin
I. Cinnamon
J. Malate
K. Turmeric
L. EGCG (Epigallocatechin Gallate).

In another preferred embodiment, any of the herein-described nutraceutical compositions of the present invention, which are formulated to be, and/or to be used as, a morning nutraceutical composition(s), a noon or midday nutraceutical composition(s), or an evening nutraceutical composition(s), of the present invention can be manufactured by mixing the respective ingredients of the same together and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, protein drinks, and/or any other edible food or beverages which can contain supplements such as those of, and/or provided by, the present invention.

In a preferred embodiment, any and/or all of the nutraceutical compositions of the present invention described herein can also be manufactured by combining, blending, or mixing, together the respective nutraceutical ingredients of the respective nutraceutical composition by mixing and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, and/or any other edible food or beverage which can contain supplements such as those of, and/or provided by, the present invention.

For example, in another preferred embodiment, any and/or all of the respective ingredients of the respective nutraceutical composition, and/or any other suitable or appropriate nutraceutical compounds or nutraceutical ingredients, can be mixed with water, or with any suitable juice or drink, in order to obtain a nutraceutical-base product for providing longevity.

As and for another example, in another preferred embodiment, any and/or all of the respective ingredients of the respective nutraceutical composition, and/or any other suitable or appropriate nutraceutical compounds or nutraceutical ingredients, can be mixed with green tea in order to obtain a nutraceutical-based green tea product for providing longevity.

As and for another example, in another preferred embodiment, any and/or all of the respective ingredients of the respective nutraceutical composition, and/or any other suitable or appropriate nutraceutical compounds or nutraceutical ingredients, can be mixed with black tea in order to obtain a nutraceutical-based black tea product for providing longevity.

As and for still another example, in another preferred embodiment, any and/or all of the respective ingredients of the respective nutraceutical composition, and/or any other suitable or appropriate nutraceutical compounds or nutraceutical ingredients, can be mixed with any type or kind of tea in order to obtain a nutraceutical-based tea product for providing longevity.

As and for yet another example, in another preferred embodiment, any and/or all of the respective ingredients of the respective nutraceutical composition, and/or any other suitable or appropriate nutraceutical compounds or nutraceutical ingredients, can be mixed with any type or kind of coffee, which can be caffeinated coffee, de-caffeinated coffee, or any flavored coffee, in order to obtain a nutraceutical-based coffee product for providing longevity.

As and for another example, in another preferred embodiment, any and/or all of the respective ingredients of the respective nutraceutical composition, and/or any other suitable or appropriate nutraceutical compounds or nutraceutical ingredients, can be mixed with any type or kind of red wine in order to obtain a nutraceutical-based red wine beverage or drink product for providing longevity.

As and for still another example, in another preferred embodiment, any and/or all of the respective ingredients of the respective nutraceutical composition, and/or any other suitable or appropriate nutraceutical compounds or nutraceutical ingredients, can be mixed with any type or kind of diet beverage or drink, nutrition beverage or drink, energy beverage or drink, protein beverage or drink, or any other type or kind of beverage or drink, in order to obtain a nutraceutical-based beverage or drink product for providing longevity.

In another preferred embodiment, any and/or all of the respective ingredients of the respective nutraceutical composition, and/or any other suitable or appropriate nutraceutical compounds or nutraceutical ingredients, can be mixed together in powder form and/or in liquid form, and/or in any combination of the same, and the resulting mixture can be used in producing a chewable food product for providing longevity.

In another preferred embodiment, any and/or all of the nutraceutical ingredients for any respective nutraceutical composition, and/or any suitable or appropriate nutraceutical compounds or nutraceutical ingredients, can also be mixed together in powder form and/or in liquid form, and/or in any combination of the same, and the resulting mixture can be used in producing a food product such as food bar, a snack bar, a cookie, a syrup, a drink or beverage additive or supplement, or a food additive or food supplement which can be in liquid form or in powdered form, for providing longevity.

In a preferred embodiment, any of the respective nutraceutical ingredients of any respective nutraceutical composition of the present invention can be mixed together by mixing and/or by processing the same in any, and/or during any, appropriate mixing process or operation, cooking process or operation, homogenization process or operation, pasteurization process or operation, and/or any other appropriate process or operation, and/or any combination of the same, which are known to those having skill in the art of the preparation of food compositions, drug compositions, food supplement compositions, chemical compositions and/or mixtures, beverages, drinks, chewable and/or edible foods, food snack bars, energy bars, energy drinks, and/or any other edible food or beverage which can contain supplements such as those of, and/or provided by, the present invention. The resulting respective nutraceutical composition can be in liquid form or in powdered form. If a powdered form is desired from a resulting liquid nutraceutical composition, an appropriate de-hydration process or operation can be performed in the container or containment system and/or in a second or other container or containment system.

Thereafter, the resulting nutraceutical composition can be in liquid form or in powdered form can be utilized in any appropriate manner, such as in producing a longevity nutraceutical-based beverage, a longevity nutraceutical-based drink, a longevity nutraceutical-based chewable food product, a longevity nutraceutical-based food product such as food bar, a snack bar, a cookie, or a syrup, a longevity nutraceutical-based drink or beverage additive or supplement, or a longevity nutraceutical-based food additive or food supplement.

In a preferred embodiment, any and/or all of the various nutraceutical compositions and/or any of the herein-described nutraceutical compositions of the present invention, which are formulated to be, and/or to be used as, a morning nutraceutical composition(s), as a noon or midday nutraceutical composition(s), or as an evening nutraceutical composition(s), of the present invention can be manufactured by, or produced by utilizing, any of the steps and/or methods described in the preferred embodiments of FIGS. 1, 2, 3, and 4, and/or by utilizing any combinations of any steps and/or methods described in the preferred embodiments of FIGS. 1, 2, 3, and 4.

In a preferred embodiment, any and/or all of the various nutraceutical compositions, and/or any of the herein-described nutraceutical compositions of the present invention, which are formulated to be, and/or which are to be used as, a morning nutraceutical composition(s), a noon or midday nutraceutical composition(s), or an evening nutraceutical composition(s), of the present invention can be manufactured by, or produced by utilizing, any of the steps and/or methods described herein and/or by using any other suitable or appropriate step(s), process(es) or method(s).

In another preferred embodiment, as well as any and/or all of the embodiments disclosed herein, the nutraceutical composition of the present invention, as well as the various nutraceutical compositions which can be formulated to be taken at different times of the day, including, but not limited to, in the morning, at noon or at midday, or in the evening, or otherwise, can also be produced so as to include any types or kinds of ingredients which are, or which can typically be, found in energy drinks, protein drinks, vitamin drinks, energy foods, protein foods, vitamin-enriched foods, energy food and/or beverage supplements, protein food and/or beverage supplements, and/or vitamin food and/or beverage supplements.

In any and/or all of the preferred embodiments described herein, any of the herein-described or herein-identified nutraceutical ingredients can be used in any of the herein-described nutraceutical compositions in, or by using, the doses or dosage ranges which are provided in Table 1, Table 2, and/or as otherwise specified in this application, and/or any dose or dosages, or dosage range or dosage ranges, which are known to those individuals who are skilled in the art as being safe doses or dosage ranges.

In a preferred embodiment, in any and/or all of the embodiments described herein and/or disclosed herein, Caffeine can have a dose or dosage range of 50 to 200 mg per serving. In a preferred embodiment, in any and/or all of the embodiments described herein and/or disclosed herein, Cacao can have a dose or dosage range of 1000 to 10,000 mg (or 1 to 10 grams) per serving. In a preferred embodiment, in any and/or all of the embodiments described herein and/or disclosed herein, Pirperine can have a dose or dosage range of 1 to 30 mg per serving.

While the present invention has been described and illustrated in various preferred and alternate embodiments, such descriptions are merely illustrative of the present invention and are not to be construed to be limitations thereof. In this regard, the present invention encompasses all modifications, variations, and/or alternate, embodiments, with the scope of the present invention being limited only by the claims which follow.

What is claimed is:

1. A nutraceutical composition, wherein a serving of the nutraceutical composition consists at least of Nicotinamide Mononucleotide (NMN) in an amount of about a range of 250 to 1000 mg, Spermidine in an amount of about a range of 0.5 to 10 mg, Rhodiola Rosea in an amount of about a range of 200 to 600 mg, Cacao in an amount of about a range of 1000 to 10,000 mg, L-Theanine in an amount of about a range of 50 to 200 mg, Glucosamine in an amount of about a range of 500 to 3000 mg, and Hyaluronic acid in an amount of about a range of 50 to 240 mg.

2. The nutraceutical composition of claim 1, wherein the nutraceutical composition further consists of Ginger in an amount of about a range of 500 to 3000 mg.

3. The nutraceutical composition of claim 1, wherein the nutraceutical composition further consists of Turmeric in an amount of about a range of 500 to 2000 mg.

4. The nutraceutical composition of claim 1, wherein the nutraceutical composition further consists of Caffeine in an amount of about a range of 50 to 200 mg.

5. A nutraceutical composition, wherein a serving of the nutraceutical composition consists at least of Epigallocatechin-3-gallate (EGCG) in an amount of about a range of 100 to 500 mg, Quercetin in an amount of about a range of 250 to 1000 mg, Resveratrol in an amount of about a range of 150 to 1000 mg, Sulforaphane in an amount of about a range of 1 to 50 mg, and Calcium Alphaketoglutarate (CaAKG) in an amount of about a range of 300 to 2000 mg.

6. The nutraceutical composition of claim 5, wherein the nutraceutical composition further consists of Fucoidan in an amount of about a range of 100-4000 mg

7. The nutraceutical composition of claim 5, wherein the nutraceutical composition further consists of Pterostilbene in an amount of about a range of 50-250 mg.

8. The nutraceutical composition of claim 5, wherein the nutraceutical composition further consists of Turmeric in an amount of about a range of 500 to 2000 mg.

9. The nutraceutical composition of claim 5, wherein the nutraceutical composition further consists of Piperine in an amount of about a range of 1 to 30 mg.

10. The nutraceutical composition of claim 5, wherein the nutraceutical composition further consists of Ginger in an amount of about a range of 500 to 3000 mg.

11. A nutraceutical composition, wherein a serving of the nutraceutical composition consists at least of Microdose Lithium in an amount of about a range of 1 to 5 mg, Trimethylglycine (TMG) in an amount of about a range of 500 to 2000 mg, Glutathione in an amount of about a range of 250 to 1000 mg, Fisetin in an amount of about a range of 100 to 1000 mg, Urolithin A in an amount of about a range of 250 to 2000 mg, Sodium Butyrate in an amount of about a range of 100-3000 mg, Berberine in an amount of about a range of 500 1500 mg, Apigenin in an amount of about a range of 50 to 500 mg, Cinnamon in an amount of about a range of 1000 to 6000 mg, and Malate in an amount of about a range of 1000 to 6000 mg.

12. The nutraceutical composition of claim 11, wherein the nutraceutical composition further consists of Turmeric in an amount of about a range of 500 to 2000 mg.

13. The nutraceutical composition of claim 11, wherein the nutraceutical composition further consists of Epigallocatechin-3-gallate (EGCG) in an amount of about a range of 100 to 500 mg.

14. The nutraceutical composition of claim 11, wherein the nutraceutical composition further consists of Cacao in an amount of about a range of 1000 to 10,000 mg.

15. The nutraceutical composition of claim 1, wherein the nutraceutical composition is produced in a powder form.

16. The nutraceutical composition of claim 5, wherein the nutraceutical composition is produced in a powder form.

17. The nutraceutical composition of claim 11, wherein the nutraceutical composition is produced in a powder form.

18. The nutraceutical composition of claim 1, wherein the nutraceutical composition is an ingredient of an energy drink, a protein drink, or a vitamin drink.

19. The nutraceutical composition of claim 5, wherein the nutraceutical composition is an ingredient of an energy drink, a protein drink, or a vitamin drink.

20. The nutraceutical composition of claim 11, wherein the nutraceutical composition is an ingredient of an energy drink, a protein drink, or a vitamin drink.
